(19) United States
(12) Patent Application Publication
(10) Pub. No.: US 2020/0208111 A1
(43) Pub. Date: Jul. 2, 2020

(54) GENOME-EDITED NK CELL AND METHODS OF MAKING AND USING

(71) Applicants: Branden S. MORIARITY, Minneapolis, MN (US); John Hunzeker, APO, AE (US); Emily Pomeroy, Minneapolis, MN (US); REGENTS OF THE UNIVERSITY OF MINNESOTA, MINNEAPOLIS, MN (US)

(72) Inventors: Branden S. Moriarity, Shoreview, MN (US); John Hunzeker, Stuttgart (DE); Emily Pomeroy, Minneapolis, MN (US)

(21) Appl. No.: 16/308,326

(22) PCT Filed: Jun. 9, 2017
(86) PCT No.: PCT/US17/36857

§ 371 Date: Dec. 7, 2018

Related U.S. Application Data
(60) Provisional application No. 62/347,668, filed on Jun. 9, 2016.

(51) Int. Cl. C12N 5/0783; C12N 15/90; C12N 9/22; C12N 15/11; C12N 15/85; A61K 35/17; A61P 35/00

(52) U.S. Cl. CPC C12N 5/0646; C12N 2502/00; C12N 9/22; C12N 15/11; C12N 15/85; A61K 35/17; A61P 35/00; C12N 2510/00; C12N 2800/80; C12N 2310/321; C12N 2310/315; C12N 2500/30; C12N 2500/05; C12N 2501/2302; C12N 15/907

(57) ABSTRACT

Described here are a genome-edited primary NK cell, methods that includes editing a genome of a primary natural killer (NK) cell, and methods of administering a genome-edited primary NK cell. The primary NK cell may be rested or stimulated.

CONTINUING APPLICATION DATA

This application is the § 371 U.S. National Stage of International Application No. PCT/US2017/036857, filed Jun. 9, 2017, which claims the benefit of U.S. Provisional Application Ser. No. 62/347,668, filed Jun. 9, 2016, the disclosures of each of which are incorporated by reference herein in their entireties.

### SEQUENCE LISTING

This application contains a Sequence Listing electronically submitted to the United States Patent and Trademark Office via EFS-Web as an ASCII text file entitled “110-05470201_ST25.txt” having a size of 8 kilobytes and created on Jun. 9, 2017. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY OF THE INVENTION

In one aspect this disclosure describes a method that includes editing a genome of a primary natural killer (NK) cell. In some embodiments, the primary NK cell includes a CD3⁻CD56⁺ cell. In some embodiments, the primary NK cell includes a stimulated NK cell.

In some embodiments, the method includes electroporation of the primary NK cell. In some embodiments, electroporation of the primary NK cell includes exposing the primary NK cell to at least 1700 volts and up to 2000 volts and at least 2 energy pulses. In some embodiments, at least one energy pulse has a duration of at least 2 milliseconds.

In some embodiments, the method includes introducing a nuclease or a nucleic acid encoding a nuclease into the primary NK cell. In some embodiments, the nuclease includes Cas9.

The method may also include introducing a chemically modified guide RNA (gRNA) into the primary NK cell. In some embodiments, the chemically modified guide RNA includes 2′-O-methyl (M), 2′-O-methyl-3′-phosphorothioate (MS), or 2′-O-methyl-3′-thiophosphonoacetate (MSP).

In some embodiments, the method includes exposing a primary NK cell to a cytokine to produce a stimulated primary NK cell. In some embodiments, the cytokine includes a cytokine bound to an artificial antigen presenting cell (aAPC).

In some embodiments, editing the genome includes editing a gene for at least one of an activating receptor, an inhibitory receptor, an adaptor molecule, a downstream signaling molecule, a component of a cytotoxic granule, a cytokine, a chemokine, a cytokine receptor, and a chemokine receptor. In some embodiments, editing the genome includes editing an ADAM17-cleavage region of CD16. In some embodiments, editing the genome includes editing a noncoding region of the genome.

In another aspect this disclosure describes a genome-edited primary NK cell. In some embodiments, the NK cell includes a CD3⁻CD56⁺ cell. In some embodiments, a gene is deleted and/or includes a point mutation. In some embodiments, the cell includes an exogenous gene.

In some embodiments, the genome-edited primary NK cell includes a modification that alters expression or activity of at least one of an activating receptor, an inhibitory receptor, an adaptor molecule, a downstream signaling molecule, a component of a cytotoxic granule, a cytokine, a chemokine, a cytokine receptor, and a chemokine receptor. In some embodiments, the NK cell includes a modification that alters expression or activity of CD16.

In some embodiments, the genome-edited primary NK cell exhibits increased stimulation-induced cytokine production, increased capacity to kill cancer cells, increased survival, and/or increased capacity to expand relative to a non-genome-edited primary NK cell.

In some embodiments, the genome-edited primary NK cell exhibits increased expression of an activating receptor relative to a non-genome-edited primary NK cell. In some embodiments, the genome-edited primary NK cell exhibits decreased expression of an inhibitory receptor relative to a non-genome-edited primary NK cell.

In a further aspect this disclosure describes a method for treating or preventing a disease in a subject, the method including administering to the subject a composition comprising a genome-edited primary NK cell as described herein. In some embodiments, the disease includes cancer, a precancerous condition, an infection with a pathogen, and/or a viral infection.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1(A-B) shows delivery of EGFP mRNA to primary human NK cells. FIG. 1A. Representative histograms depicting flow cytometry analysis of viability (left, measured with APC e-Fluor 780 Fixable Viability Dye) and EGFP expression (right) of unstimulated primary human NK cells 48 hours post electroporation with EGFP encoding mRNA. FIG. 1B. Average viability and EGFP expression from four independent donors.

FIG. 2 shows CRISRP/Cas9 nuclease activity at CCR5 and PD1 in primary human NK cells. Representative Surveyor nuclease activity detected successful gene editing at CCR5 (left) and PD1 (right) 72 hours post electroporation.

FIG. 3 shows the results of electroporating 3 million unstimulated NK cells/group with 10 μg EGFP mRNA (TriLink BioTechnologies, San Diego, Calif.) using either the AMAXA platform (Macrophage kit) or the NEON platform (T cell protocol). GFP was measured 48 hours after electroporation. Viability with the AMAXA platform was 62.5%; viability with the NEON platform was 79.1%.

FIG. 4 shows GFP mRNA Expression in unstained NK cells and NK cells isolated from frozen (Donor 10) or fresh (Trima Cone) PBMC. Three million unstimulated NK cells were electroporated with the AMAXA platform (Macrophage kit). GFP was measured 48 hours after electroporation. Viability in NK cells isolated from frozen and fresh PBMC was 86% and 60%, respectively.

FIG. 5 shows CRISRP/Cas9 nuclease activity at CCR5 in unstimulated primary human NK cells three days after electroporation. Unstimulated cells were electroporated with GFP (10 μg) or Cas9. The Surveyor nuclease activity assay depicts successful gene editing at CCR5 locus after electroporation using the AMAXA platform but not the NEON platform (T cell protocol). (PC=positive control.)

FIG. 6 shows GFP expression in primary NK cells expanded using membrane-bound IL-21 (Clone 9) cells. Three million cells were electroporated using the AMAXA platform (Macrophage kit) or the NEON platform (T cell protocol). GFP expression was measured 48 hours after electroporation.

FIG. 7 shows CRISRP/Cas9 nuclease activity at CCR5 in primary human NK cells expanded using artificial antigen-presenting cells (aAPCs) expressing membrane-bound IL-21 (Clone 9) cells prior to electroporation. Cells (3×10⁶) were electroporated (using the AMAXA plastform) with Cas9 (15 μg) alone or Cas9 mRNA (15 m) and CCR5 gRNA (10m), and DNA was harvested 3 days after electroporation. The Surveyor nuclease activity assay detected successful gene editing at CCR5 locus but with lower efficiency compared to the same locus in cells that were not expanded using Clone 9 cells prior to electroporation.

FIGS. 8(A-C) shows optimization of electroporation conditions using the NEON Transfection system including the cell count (FIG. 8A), the percentage of cells that were GFP⁺ (FIG. 8B), and the mean fluorescence intensity (MFI) (FIG. 8C). The electroporation conditions on the X-axis are further enumerated in Table 3. The electroporation conditions indicated with an arrow were selected for use in additional experiments.

FIGS. 9(A-B) shows CISH expression in was decreased in NK cells electroporated with CISH gRNA and Cas9 mRNA compared to matched donor samples electroporated with Cas9 mRNA alone; the effect of stimulation with IL-15 on electroporated cells was also examined. FIG. 9A. Five days after electroporation, cells were harvested and protein lysates were run with CISH-specific antibody on a Wes machine (Protein Simple, San Jose, Calif.). Results are quantified in FIG. 9B; analyzed by student's t-test, n=3 donors, error bars=1 s.d., *P=0.0333 (comparing Cas9 24 hr IL-15 to CISH 24 hr IL-15), **P=0.0083 (comparing Cas9 No stim to CISH No stim).

FIG. 10 shows the percent editing of cells electroporated with 15 μg Cas9 mRNA and 15 μg gRNA (CISH, PD1, ADAM17, or TIGIT) compared to matched donor samples electroporated with 15 μg Cas9 mRNA alone. n=3 donors for CISH and PD1; n=2 donors for ADAM17 and TIGIT.

FIGS. 11(A-B) shows TIGIT expression in knockout cells was decreased in cells electroporated with TIGIT gRNA and Cas9 mRNA compared to matched donor samples electroporated with Cas9 mRNA alone. FIG. 11A shows percent of cells expressing TIGIT; left panels show cells electroporated with Cas9 mRNA alone; right panels show cells electroporated with TIGIT gRNA and Cas9 mRNA; top panels show donor 407; bottom panels show donor 613. Results are quantified in FIG. 11B; analyzed by student's t-test, n=2 donors, error bars=1 s.d., *P=0.0128.

FIGS. 12(A-D) shows exemplary CD16 expression in NK cells electroporated with 15 μg Cas9 mRNA and 15 μg ADAM17 gRNA; five days after electroporation, cells were treated for 1 hour with an ADAM17 inhibitor or DMSO; cells were then stimulated for 1 hour with 1 μg/mL PMA or left unstimulated, as described in Example 2D. FIG. 12A shows exemplary flow cytometry plots of electroporated NK cells (from donor 407) treated with DMSO and stimulated for 1 hour with 1 μg/mL PMA (right panel) or left unstimulated (left panel). FIG. 12B shows exemplary flow cytometry plots of electroporated NK cells (from donor 407) treated with 1 μM (left panel) or 10 μM (right panel) of ADAM17 inhibitor, and stimulated for 1 hour with 1 μg/mL PMA. FIG. 12C shows exemplary flow cytometry plots of unstimulated (left panel) or PMA-stimulated (right panel) electroporated NK cells (from donor 407) treated with an ADAM17 inhibitor. Results from two donors are quantified in FIG. 12D; analyzed by student's t-test, n=2 donors, error bars=1 s.d., ***P=0.0003 (comparing Cas9 mRNA alone compared to Cas9+ADAM17 guide RNA).

FIGS. 13(A-C) shows GFP expression in NK cells 5 days after electroporation using donor vector that expressed GFP after homologous recombination into the AAVS1 site (Doggybone Splice-Acceptor GFP). FIG. 13A shows GFP expression in NK cells electroporated with 15 μg of Cas9 and no guide RNA (left panel) or with 15 μg of Cas9 and with AAVS1 gRNA (right panel). FIG. 13B shows GFP expression in NK cells electroporated with 1 μg of vector (left panel) or with 1 μg of vector and with 15 μg of Cas9 and AAVS1 gRNA (right panel). FIG. 13C shows GFP expression in NK cells electroporated with 10 μg of vector (left panel) or with 10 μg of vector and with 15 μg of Cas9 and AAVS1 gRNA (right panel).

FIG. 14 shows donor GFP expression was detected by flow cytometry 12 days after electroporation of NK cells.

FIG. 15 shows vector DNA was stably integrated into NK cells, as determined by junction PCR.

FIG. 16 shows GFP expression in NK cells 12 days after electroporation with and without 10 μg of a DNA donor vector that expressed GFP after homologous recombination into the AAVS1 site (Minicircle Splice Acceptor GFP).

### DETAILED DESCRIPTION

Natural killer (NK) cells are cytotoxic lymphocytes capable of human immune surveillance. Although NK cells offer a potential source of cells for cancer immunotherapy, their success in the clinic has been limited. Compared to other lymphocytes (like T cells), the ability to edit the genome of a natural killer cell has proved especially elusive. Such editing would enhance the ability to use NK cells in immunotherapy. This disclosure describes genome-edited primary NK cells, methods of making those cells, and methods of administering those cells.

A primary NK cell may express CD16 and/or CD56. In some embodiments, an NK cell does not express CD3. In some embodiments, an “NK cell” is preferably defined as a cell that is CD56⁺ and CD3⁻. In some embodiments, an “NK cell” is defined as a cell that is CD16⁺ and CD3⁻.

NK cells are lymphocytes of the innate immune system that kill virally infected or transformed cells. Like T cells, NK cells are cytotoxic lymphocytes. Unlike T cells, NK cells do not require antigen recognition, and require integration of signals from many activating and inhibitory receptors to perform their function. Despite their similarities to T cells, NK cells behave differently under stimulation conditions and do not tolerate electroporation in the same way as T cells.

A primary NK cell may be isolated from, for example, peripheral blood, umbilical cord cells, ascites, and/or a solid tumor. In some embodiments, a “primary NK cell” is an NK cell that is freshly isolated. In some embodiments, a “primary NK cell” is an NK cell that has undergone up to 5 replications or divisions after being isolated, up to 10 replications or divisions after being isolated, up to 15 replications or divisions after being isolated, up to 20 replications or divisions after being isolated, up to 25 replications or divisions after being isolated, up to 30 replications or divisions after being isolated, up to 35 replications or divisions after being isolated, or up to 40 replications or divisions after being isolated. In some embodiments, the primary NK cell is a non-clonal cell. In some embodiments, primary NK cell is a proliferating cell. In some embodiments, primary NK cell is an expanded cell. A primary NK cell is preferably not derived from an induced pluripotent stem cell (iPSC).

In some embodiments, the NK cell is a mammalian cell. In some embodiments, the NK cell is preferably a human cell. In some embodiments, the NK cell is a mouse cell.

A primary NK cell is “genome-edited” if the primary NK cell includes a modification to the genome compared to a non-genome-edited NK cell. In some embodiments, a non-genome-edited NK cell is a wild type NK cell. In some embodiments, a non-genome-edited NK cell may be a freshly isolated NK cell.

In some embodiments, the genome-edited primary NK cell includes a modifying a noncoding region of the genome and/or a coding region of the genome (for example, a gene). In some embodiments, the noncoding region of the genome may include a sequence for a small, regulatory noncoding RNA, including, for example, a microRNA (miRNA). In some embodiments, the noncoding region of the genome is preferably involved in regulating the function, activation, and/or survival of the NK cell.

In some embodiments, a portion of genomic information and/or a gene may be deleted. In some embodiments, a portion of genomic information and/or a gene may be added. In some embodiments, the genomic information and/or the gene that is added is exogeonous. In some embodiments, “exogenous” genomic information or an “exogenous” gene may be genomic information or a gene from a non-NK cell. In some embodiments, “exogenous” genomic information or an “exogenous” gene may be an additional copy of genomic information or a gene already present in the NK cell. In some embodiments, “exogenous” genomic information or an “exogenous” gene may be genomic information or a gene from a cell of another species than the NK cell being modified. In some embodiments, “exogenous” genomic information or an “exogenous” gene may be artificially generated including, for example, nucleic acids encoding a chimeric antigen receptor (CAR) or a marker gene. In some embodiments, a portion of genomic information and/or a gene may be altered, for example, by a mutation. A mutation may include, for example, a point mutation, a frameshift mutation, etc.

In some embodiments, a genome-edited primary NK cell preferably includes a modification that alters expression or activity of the genome-edited primary NK cell relative to a non-genome-edited primary NK cell. For example, in some embodiments, the genome-edited primary NK cell may exhibit increased antibody-dependent cell cytotoxicity (ADCC) relative to a non-genome-edited primary NK cell. In some embodiments, the genome-edited primary NK cell may exhibit increased capacity to kill cancer cells relative to a non-genome-edited primary NK cell.

In some embodiments, the genome-edited primary NK cell preferably includes a modification that alters survival of the genome-edited primary NK cell relative to a non-genome-edited primary NK cell. In some embodiments, the genome-edited primary NK cell exhibits increased capacity to expand relative to a non-genome-edited primary NK cell. The expansion may be, for example, in vivo or in vitro. In some embodiments, the expansion may be in vitro after co-culturing with a cytokine, a cancer cell line, or both.

In some embodiments, the genome-edited primary NK cell may include a modification that alters cytokine or chemokine production relative to a non-genome-edited primary NK cell. In some embodiments, the cytokine or chemokine production may be stimulation-induced. Cytokines and chemokines could include, for example, IFNγ, TNFα, IL-17, IL-22, MIP-1α (CCL3), MIP-1β (CCL4), and/or RANTES (CCLS). Such a modification could include, for example, an alteration of the transcription process of the gene encoding the cytokine or chemokine, or the alteration or deletion of a negative regulator of cytokine or chemokine production (for example, cytokine-inducible SH2-containing protein (CISH)). In some embodiments, the genome-edited primary NK cell may include a modification of a cytokine receptor. Cytokine receptors could include, for example, IL-2R, IL-12R, IL-15R, IL-18R, IL-21R, etc. Such a modification could include, for example, an alteration of the transcription process of the gene encoding the cytokine receptor, or the alteration or deletion of a regulatory portion of the cytokine receptor.

In some embodiments, the genome-edited primary NK cell may include a modification of a chemokine receptor. Chemokine receptors could include, for example, CCR1, CCR2, CCR3, CCR4, CCR5, CCR6, CCR7, CXCR1, CXCR2, CXCR3, CXCR4, CXCR5, CXCR6, etc. Such a modification could include, for example, an alteration of the transcription process of the gene encoding the chemokine receptor, or the alteration or deletion of a regulatory portion of the chemokine receptor.

In some embodiments, the genome-edited primary NK cell includes a modification that alters one or more elements of a cytotoxic granule. The elements of the cytotoxic granule may include components contained in the granule including, for example, Granzyme B and/or perforin. The elements of the cytotoxic granule may additionally or alternatively include a protein with a function in the exocytosis of a cytotoxic granule including, for example, Wiskott-Aldrich Syndrome protein (WASp), WASp Interacting protein (WIP), Cdc42 Interacting protein-4 (CIP), Adaptor protein 3 complex (AP-3), Rab7 interacting lysosomal protein (RILP)/Rab7, Rab27a, Myosin IIa, Munc13-4, Syntaxin 11, VAMP7, Syntaxin 7, and/or Dynamin 2.

In some embodiments, the genome-edited primary NK cell includes a modification that alters expression or activity of an activating receptor relative to a non-genome-edited primary NK cell. For example, expression of the activating receptor may be increased. In some embodiments, the activating receptor includes CD16, IL-15 receptor (IL-15R), CD94-NKG2C, NKG2D, 2B4, DNAM-1 (CD226), a member of the KIR2DS family (including, for example, KIR2DS1, KIR2DS2, KIR2DS3, KIR2DS4, and KIR2DS5), a member of the KIR3DS family (including, for example, KIR3DS1), NKG2C, NKG2D, NKG2E, PILR (CD99), NKp30, NKp44, NKp46, NKp80, Sema4D (CD100), and/or CD160.

In some embodiments, the genome-edited primary NK cell includes a modification that alters expression or activity of CD16. For example, a modification may render CD16 hyperactive. A modification could, alternatively or additionally, alter the intracellular domains of CD16. In some embodiments, a modification of CD16 could include fusion of CD16 or a component of CD16 to other components such as those found in Bispecific Killer Engagers (BiKEs) or Trispecific Killer Engagers (TriKEs). (Carlsten et al., Frontiers in Immunology, 2015, 6:266; Vallera, D. A. et al. Clinical Cancer Research, Feb. 4, 2016, pii: clincanres.2710.2015.) In some embodiments, a modification of CD16 may include an alteration to the ADAM17 cleavage region. For example, a modification could render CD16 resistant to ADAM17-mediated proteolytic cleavage (Jing et al., PLoS ONE, 2015, 10(3):e0121788, doi: 10.1371/journal.pone.0121788).

In some embodiments, the genome-edited primary NK cell includes a modification that alters expression or activity of an inhibitory receptor relative to a non-genome-edited primary NK cell. For example, expression of the inhibitory receptor may be decreased.

In some embodiments, the inhibitory receptor includes PD-1, CD94-NKG2A, NKG2A, TIGIT, CISH, a member of the KIR2DL family (for example, KIR2DL1; KIR2DL2; KIR2DL3; KIR2DL4; or KIRDL5), a member of the KIR3DL family (KIR3DL1; KIR3DL2; or KIR3DL3), KLRG1, LILR, 2B4 (CD48), CD96 (Tactile), LAIR1, KLB1 (CD161), CEACAM-1, SIGLEC3, SIGLEC7, SIGLEC9, and/or CTLA4.

In some embodiments, the genome editing primary NK cell includes a modification that alters expression or activity of an adaptor molecule and/or a downstream signaling molecule. In some embodiments, the adaptor molecule may include EAT2, DAP10, DAP12, and/or CD3zeta.

In some embodiments, the genome-edited primary NK cell includes a modification that introduces a non-endogenous gene including, for example, a marker gene (also referred to as a reporter gene) such as GFP, enhanced GFP (EFGP), etc.

This disclosure also describes a method of making a genome-edited primary NK cell.

In some embodiments, the method includes a technique to introduce a protein or nucleic acid into the primary NK cell. Any suitable method of introducing a protein or nucleic acid may be used. In some embodiments, the method preferably includes electroporation of a primary NK cell to introduce genetic material including, for example, DNA, RNA, and/or mRNA. In some embodiments a technique to introduce a protein or nucleic acid may include introducing a protein or nucleic acid via electroporation; microinjection; viral delivery; exosomes; liposomes; biolistics; jet injection; hydrodynamic injection; ultrasound; magnetic field-mediated gene transfer; electric pulse-mediated gene transfer; use of nanoparticles including, for example, lipid-based nanoparticles; incubation with a endosomolytic agent; use of cell-penetrating peptides; etc.

In some embodiments, the method includes electroporation of a primary NK cell to introduce a protein or a nucleic acid (for example, DNA, RNA, and/or mRNA).

In some embodiments, the method includes electroporation of a primary NK cell using an AMAXA nucleoporator and/or the AMAXA Human Macrophage Cell Nucleofector Kit (Lonza, Switzerland). In some embodiments, the use of AMAXA Program Y-010 is preferred.

In some embodiments, the method includes electroporation of a primary NK cell including, for example, using an NEON transfection system (Thermo Fisher Scientific Inc., Waltham, Mass.). The electroporation method may include any method determined to be suitable to a skilled artisan.

For example, in some embodiments, electroporation may include exposing the primary NK cell to at least 1700 volts, at least 1750 volts, at least 1800 volts, at least 1850 volts, at least 1900 volts, at least 1950 volts, at least 2000 volts, at least 2050 volts, at least 2100 volts, or at least 2150 volts. In some embodiments, electroporation may include exposing the primary NK cell to up to 1850 volts, up to 1900 volts, up to 1950 volts, up to 2000 volts, up to 2050 volts, up to 2100 volts, up to 2150 volts, up to 2200 volts, or up to 2250 volts. For example, in some embodiments, a stimulated primary NK cell may be exposed to between 1750 and 1950 volts. In some embodiments, a unstimulated primary NK cell may be exposed to between 2100 and 2200 volts.

In some embodiments, electroporation may include exposing the primary NK cell to multiple pulses of energy. For example, electroporation may include exposing the primary NK cell to at least 1 energy pulse, at least 2 energy pulses, at least 3 energy pulses, at least 4 energy pulses, or at least 5 energy pulses. In some embodiments, the primary NK cell may be exposed to up to 2 energy pulses, up to 3 energy pulses, up to 4 energy pulses, up to 5 energy pulses, up to 6 energy pulses, or up to 10 energy pulses.

The electroporation may include exposing the primary NK cell an energy pulse or multiple pulses for any suitable length of time. For example, a pulse may last at least 2 milliseconds, at least 3 milliseconds, at least 4 milliseconds, at least 5 milliseconds, at least 7 milliseconds, at least 9 milliseconds, at least 10 milliseconds, at least 20 milliseconds, at least 30 milliseconds, or at least 40 milliseconds. In some embodiments, at pulse may last up to 8 milliseconds, up to 10 milliseconds, up to 12 milliseconds, up to 15 milliseconds, up to 20 milliseconds, up to 30 milliseconds, up to 40 milliseconds, or up to 50 milliseconds.

In some embodiments, the primary NK cells at the time of electroporation and/or transfection are unstimulated cells (sometimes also referred to as rested cells or resting cells), that is, the cells that have not been subjected to an activation or proliferation step. In some embodiments, an unstimulated NK cell may include a cell that has been incubated overnight in BO media +1 ng/mL IL-15. In some embodiments, stimulating a primary NK cell prior to introducing a EGFP mRNA into the primary NK cell resulted in lower EGFP expression compared to introducing a EGFP mRNA into unstimulated cells.

In some embodiments, the primary NK cells at the time of electroporation and/or transfection are preferably stimulated cells. Stimulated cells may include cells that have been subjected to conditions whereby the cell is transitioned from a resting state to an active or stimulated state. In some embodiments, stimulated cells have been subjected to an activation and/or proliferation step. In some embodiments, a stimulated NK cell includes an expanded NK cell.

In some embodiments, the primary NK cell may be additionally or alternatively stimulated after being electroporated and/or transfected. For example, the NK cell may be stimulated beginning immediately after, one day after, two days after, three days after, four days after, five days after, six days after, seven days after, eight days after, nine days after, and/or 10 days after electroporation.

An NK cell may be stimulated using any suitable method and for any suitable length of time. In some embodiments, a stimulated NK cell includes an NK cell exposed to phorbol-12-myristate-13-acetate (PMA). In some embodiments, a stimulated NK cell includes an NK cell exposed to cytokines including, for example, IL-21, IL-2, IL-12, IL-15, type I interferons, etc. In some embodiments, the cytokine may include a soluble cytokine. In some embodiments, the cytokines are bound cytokines. In some embodiments, the cytokines may be bound to a surface (including, for example, the surface of a tissue culture flask).

In some embodiments, a bound cytokine may be bound to an artificial antigen presenting cell (aAPC). An aAPC can include, for example, clone 9, described by Denman et al., PLoS One, 2012, 7(1): e30264 doi: 10.1371/journal.pone.0030264. In some embodiments, an aAPC may be a bead. A spherical polystyrene bead may be coated with antibodies against NK cell surface proteins and be used for NK cell activation. A bead may be of any size. In some cases, the bead may be or may be 3 and 6 micrometers. A bead may be 4.5 micrometers in size. A bead may be utilized at any cell to bead ratio. For example, a 3 to 1 bead to cell ratio at 1 million cells per milliliter may be used. In some embodiments, an aAPC may be a rigid spherical particle, a polystyrene latex microbeads, a magnetic nano- or micro-particle, a nanosized quantum dot, a poly(lactic-co-glycolic acid) (PLGA) microsphere, a nonspherical particle, a carbon nanotube bundle, an ellipsoid PLGA microparticle, a nanoworm, a fluidic lipid bilayer-containing system, a 2D-supported lipid bilayer (2D-SLBS), a liposome, a RAFTsomes/microdomain liposome, an supported lipid bilayer particle, or any combination thereof.

In some embodiments, a stimulated NK cell includes an NK cell treated with a commercially available kit including, for example, CellXVivo Human NK Cell Expansion Kit (R&D Systems, Minneapolis, Minn.), Human NK Cell Expansion Activator Kit (Miltenyi Biotech, Bergisch Gladbach, Germany), etc.

In some embodiments, a stimulated NK cell includes an NK cell in a population that has been expanded at least 3 fold, at least 4 fold, at least 5 fold, at least 6 fold, at least 7 fold, or at least 8 fold. In some embodiments, a stimulated NK cell includes an NK cell in a population that has been expanded up to 5 fold, up to 6 fold, up to 7 fold, up to 8 fold, up to 10 fold, up to 20 fold, or up to 30 fold.

In some embodiments, the NK cells may be stimulated hours. In some embodiments, the NK cell may be stimulated for days. For example, in some embodiments, an NK cell may be co-cultured with an aAPC for up to 1 day, up to 2 days, up to 3 days, up to 4 days, up to 5 days, up to 6 days, up to 7 days, up to 8 days, or up to 9 days, up to 2 weeks, up to 3 weeks, and so forth.

In some embodiments, the method includes introducing a nuclease or nucleic acids encoding a nuclease. A nuclease may include, for example, a targeted nuclease. A nuclease may include, for example, an RNA-guided endonuclease (RGEN) including, for example, Cas9; a transcription activator-like effector nuclease (TALEN); a zinc-finger nuclease (ZFN), etc. The nuclease and/or components of the nuclease system (including, for example, CRISPR) may be introduced in any suitable form including, for example, as DNA, as RNA, as mRNA, in a plasmid, as a protein, etc.

In some embodiments, the method preferably includes inducing double stranded breaks in the genome of the primary NK cell using a CRISPR system (for example, a CRISPR/Cas9 system). In some embodiments, the method preferably includes introducing CRISPR, a CRISPR nuclease (including, for example, Cas9 and/or Cpfl) or DNA or RNA encoding CRISPR and a CRISPR nuclease (including, for example, DNA or RNA encoding Cas9 or Cpf1). The method can, in some embodiments, include introducing a guide RNA (gRNA).

In some embodiments, the method may include homologous recombination including, for example, Cas9-triggered homologous recombination. For example, Cas9 may be used to introduce a DNA double-strand break at a defined site. At the same time, a homologous repair template including a genome modification may be introduced. When the double-strand break is repaired by homologous recombination with the modified template, insertions, deletions, point mutants, in-frame GFP fusions, and other modifications may be introduced. The ability induce homologous recombination in primary NK cells was unexpected because DNA is toxic to primary cells, and successful homologous recombination in NK cells had not been previously reported.

In some embodiments, the gRNA preferably includes a chemically modified gRNA. In some embodiments, the chemical modification to the gRNA preferably decreases a cell's ability to degrade the RNA. In some embodiments, a chemically modified gRNA includes one or more of the following modifications: 2′-fluoro (2′—F), 2′-O-methyl (2′-O—Me), S-constrained ethyl (cEt), 2′-O-methyl (M), 2′-O-methyl-3′-phosphorothioate (MS), and/or 2′-O-methyl-3′-thiophosphonoacetate (MSP). In some embodiments, the chemically modified gRNA may include a gRNA and/or a chemical modification described in Hendel et al, Nature Biotechnology, 2015, 33(9):985-989 or Randar et al., PNAS, 2015, 112(51):E7110-7.

The gRNA target may include, for example, any suitable target. In some embodiments, the gRNA target includes a portion of the NK genome including, for example, a gene or a portion of a gene. For example, a gRNA target may include a cytokine, a chemokine, a cytokine and/or chemokine receptor, an NK cell activating receptor, an NK cell inhibitory receptor, an adaptor molecule, and/or a downstream signaling molecule. Additionally or alternatively, a gRNA target may include a portion of a cytokine, a portion of a chemokine, a portion of a cytokine and/or chemokine receptor, a portion of an NK cell activating receptor, a portion of an NK cell inhibitory receptor, a portion of an adaptor molecule, and/or a portion of a downstream signaling molecule.

In some embodiments, the method includes introducing a DNA-guided DNAse. In some embodiments, the method includes introducing Natronobacterium gregoryi Argonaute (NgAgo). In some embodiments, NgAgo may be used as a DNA-guided endonuclease. (Gao et al., Nature Biotechnology, 2016, doi:10.1038/nbt.354.) The method may further include, for example, introducing a guide DNA (gDNA).

In some embodiments, the method includes editing a gene. Editing a gene may include introducing one or more copies of the gene, altering the gene, deleting the gene, upregulating expression of the gene, downregulating expression of the gene, mutating the gene, methylating the gene, demethylating the gene, acetylating the gene, and/or deacetylating the gene. Mutating the gene may include introducing activing mutations, introducing inactivating and/or inhibitory mutations, and/or introducing point mutations. Editing the gene can, additionally or alternatively, include modification the genomic sequence to include additional activating components including components such as a chimeric antigen receptor and/or a component found in a Bispecific Killer Engager (BiKE) or a Trispecific Killer Engager (TriKE). (Carlsten et al., Frontiers in Immunology, 2015, 6:266; Vallera, D. A. et al. Clinical Cancer Research, Feb. 4, 2016, pii: clincanres.2710.2015.)

In some embodiments, the method includes editing a gene for an activating receptor. In some embodiments, the activating receptor/molecule includes CD16, IL-15 receptor, CD94-NKG2C, NKG2D, 2B4, DNAM-1 (CD226), a member of the KIR2DS family (including, for example, KIR2DS1, KIR2DS2, KIR2DS3, KIR2DS4, and KIR2DS5), a member of the KIR3DS family (including, for example, KIR3DS1), NKG2C, NKG2D, NKG2E, PILR (CD99), NKp30, NKp44, NKp46, NKp80, Sema4D (CD100), and/or CD160. Editing the gene for an activating receptor may include introducing an activating mutation and/or a mutation that increase expression and/or activity of the activating receptor.

In some embodiments, the method includes editing a gene for an inhibitory receptor. In some embodiments, the inhibitory receptor includes PD-1, CD94-NKG2A, TIGIT, CISH, NKG2A, a member of the KIR2DL family (for example, KIR2DL1; KIR2DL2; KIR2DL3; KIR2DL4; or KIRDL5), a member of the KIR3DL family (KIR3DL1; KIR3DL2; or KIR3DL3), KLRG1, LILR. 2B4 (CD48), CD96 (Tactile), LAIR1, KLB1 (CD161), CEACAM-1, SIGLEC3, SIGLEC7, SIGLEC9, and/or CTLA4. In some embodiments, editing the gene for an inhibitory receptor may include introducing an inactivating mutation and/or a mutation that decreases expression and/or activity of the inhibitory receptor.

In some embodiments, the method includes editing a gene for an adaptor molecule. In some embodiments, the adaptor molecule includes EAT2, DAP10, DAP12, and/or CD3zeta.

In some embodiments, the method includes editing a gene for a cytokine or chemokine. In some embodiments, the cytokine or chemokine includes, for example, IFNγ, TNFα, IL-17, IL-22, MIP-1α (CCL3), MIP-1β (CCL4), and/or RANTES (CCL5).

In some embodiments, the method includes editing a gene for a cytokine receptor. Cytokine receptors could include, for example, IL-2R, IL-12R, IL-15R, IL-18R, and/or IL-21R.

In some embodiments, the method includes editing a gene for a chemokine receptor. Chemokine receptors could include, for example, CCR1, CCR2, CCR3, CCR4, CCRS, CCR6, CCR7, CXCR1, CXCR2, CXCR3, CXCR4, CXCRS, and/or CXCR6.

In some embodiments, the method includes editing a gene for a component of a cytotoxic granule. The component of the cytotoxic granule may include components contained in the granule including, for example, Granzyme B and/or perforin. The component of the cytotoxic granule may additionally or alternatively include a protein with a function in the exocytosis of a cytotoxic granule including, for example, Wiskott-Aldrich Syndrome protein (WASp), WASp Interacting protein (WIP), Cdc42 Interacting protein-4 (CIP), Adaptor protein 3 complex (AP-3), Rab? interacting lysosomal protein (RILP)/Rab7, Rab27a, Myosin IIa, Munc13-4, Syntaxin 11, VAMP7, Syntaxin 7, and/or Dynamin 2.

In some embodiments, the method includes editing a gene for a downstream signaling molecule. In some embodiments, the method includes editing a gene that regulates expression and/or function of an NK cell receptor. For example, the method may include editing a gene for a disintegrin and metalloprotease-17 (ADAM17), a protein implicated in CD16 shedding; and/or tumor necrosis factor-related apoptosis-inducing ligand (TRAIL), a cytotoxic effector molecule.

In some embodiments, the method includes introducing a non-endogenous (also referred to herein as an exogenous) gene. In some embodiments, homologous recombination may be used to introduce a non-endogenous gene. In some embodiments, the non-endogenous gene may include a marker gene (e.g., GFP, EGFP, etc.). In some embodiments, the non-endogenous gene may include a chimeric antigen receptor (CAR). in some embodiments, an exogenous gene may be inserted at adeno-associated virus integration site 1 (AAVS1).

In some embodiments, the method includes editing a noncoding region of the genome. For example, the method may include editing a sequence for a small, regulatory noncoding RNA, including, for example, a microRNA (miRNA). In some embodiments, the noncoding region of the genome is preferably involved in regulating the function, activation, and/or survival of the NK cell.

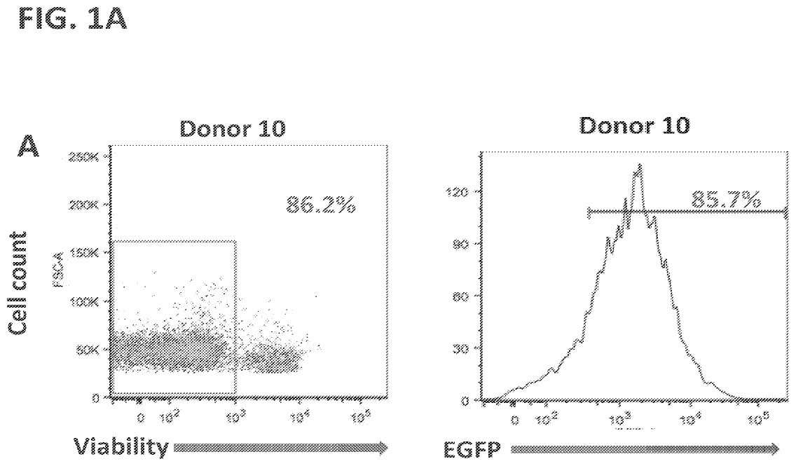

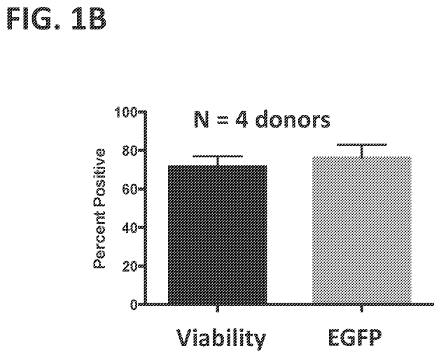

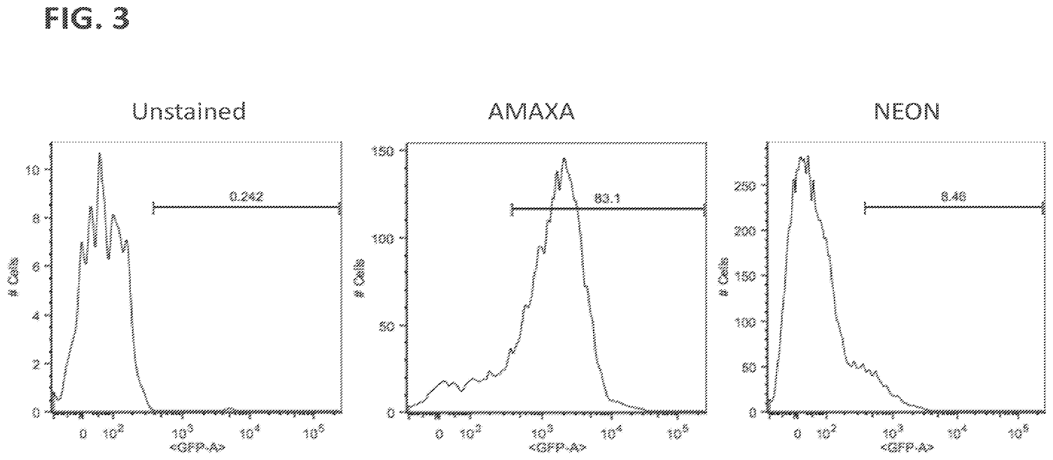

Surprisingly, the method described herein has proved effective for genome editing of an NK cell including introducing, deleting, or altering a gene from primary NK cells. For example, as shown in FIG. 1 and FIG. 3, use of the AMAXA nucleoporator and/or the AMAXA Human Macrophage Cell Nucleofector Kit may be used to introduce mRNA encoding EGFP into primary NK cells. The effectiveness of the AMAXA Human Macrophage Cell Nucleofector Kit—indicated for use with a different cell type—for electroporation of NK cells was unexpected.

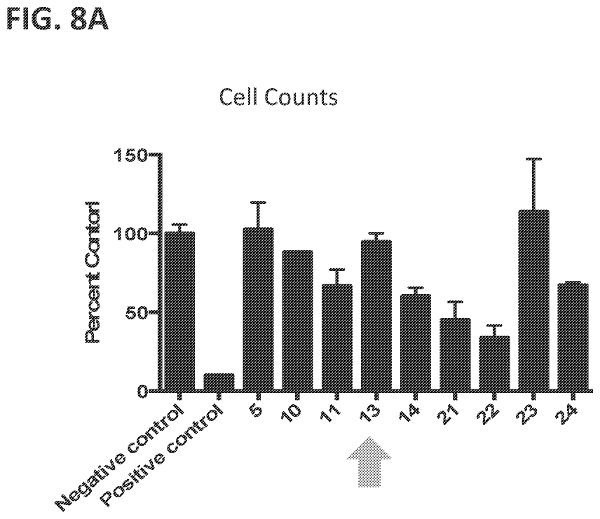

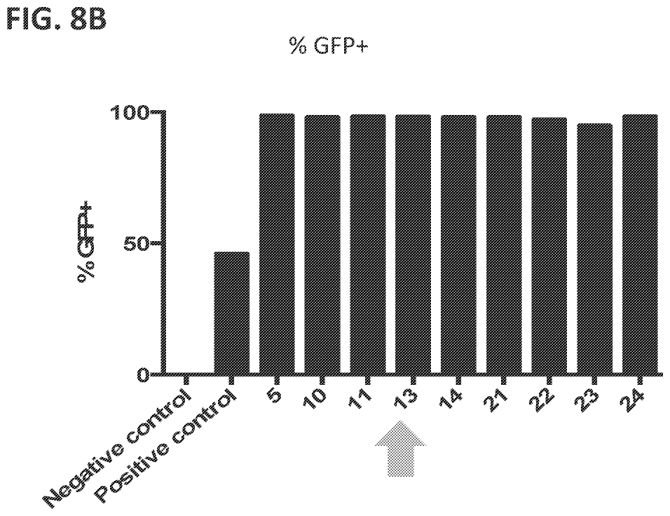

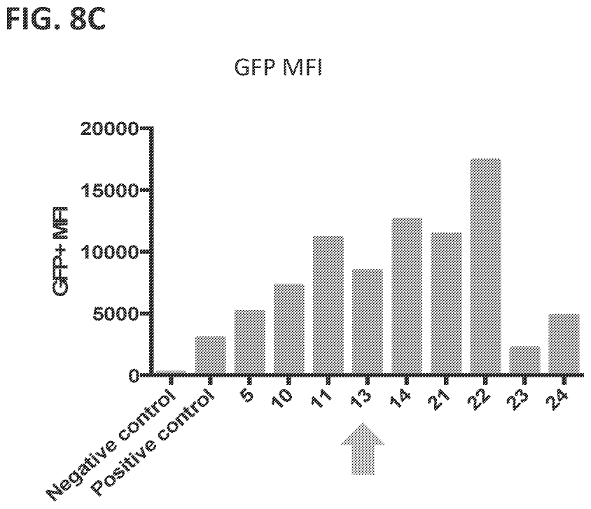

Moreover, while use of the AMAXA nucleofector resulted in increased rate of EGFP expression over electroporation with a NEON platform for unstimulated primary NK cells, development and optimization of an electroporation protocol for stimulated primary NK cells using a NEON electroporation system resulted in even higher rates of EGFP expression and improved cell viability, as shown in FIG. 8. These results were surprising at least because a electroporation protocol using a NEON platform had not been previously shown to be effective for rested NK cells.

Additional testing indicated that rested NK cells could also be successfully transfected using the NEON platform. Because NK cells are lymphocytes like T cells, electroporation conditions that worked in T cells were tested initially. At the voltages successful for transfecting T cells (approximately 1400 volts), no GFP expression was seen in NK cells. These results were surprising, as it could have been predicted that NK cells would require the same electroporation conditions as T cells. Many additional electroporation conditions were tried, and the voltage was increased far beyond what was required for T cells (to at least 2200 volts). Higher voltage electroporation was found to increase transfection of NK cells but to the detriment of their viability. A wide range of voltages, pulse widths, and pulse lengths were tested to find electroporation conditions that yield high expression of GFP with very high viability. Through painstaking trial and error, as shown in FIG. 8, 2 pulses of 1850 volts of 10 milliseconds were found to provide the highest rate of nucleic acid delivery without negatively affecting viability in expanded NK cells.

The voltages required to successfully transfect the NK cells were significantly higher than what was required for T cells. For example, at least 1850 volts was needed to successful transfect NK cells compared to 1400 volts for T cells. In addition, for transfection of expanded NK cells, higher voltages were needed than for T cells but lower voltages could be used than the voltages needed for rested NK cells. For example, in some experiments, 2 pulses of 2150 volts of 10 milliseconds were found to provide the highest rate of nucleic acid delivery without negatively affecting viability in rested NK cells.

Thus, the methods described herein permit introducing, deleting, and/or altering a gene from primary NK cells. Modifying and/or deleting a gene of the NK cell genome, particularly the genome of primary NK cells, has proved particularly elusive.

In some embodiments, the method includes selecting an NK cell. In some embodiments the selection is performed after editing a gene. NK cells can, in some embodiments, be selected using one or more of the following methods: flow sorting (including, for example, for GFP expression); magnetic bead separation (including, for example, targeting a cell-surface marker); transient drug resistance gene expression (including, for example, antibiotic resistance). In some embodiments, the selection may be for an NK cell that has an edited genome.

In some embodiments, the method includes expanding an edited NK cell. In some embodiments, the expansion may be performed after selecting the NK cell. In some embodiments, an NK cell may be expanded by co-incubation with an artificial antigen-presenting cells (aAPC). In some embodiments, an NK cell may be expanded by co-incubation with an aAPC bound to a cytokine. In some embodiments, an NK cell may be expanded by co-incubation with a soluble cytokine. The cytokine may include, for example, IL-21, IL-2, IL-12, IL-15, type I interferons, etc. In some embodiments, an NK cell may preferably be expanded by co-incubation with an aAPC bound to IL-21 or expressing membrane-bound IL-21.

This disclosure further provides methods for using the genome-edited primary NK cell described herein. For example, a genome-edited primary NK cells may be used to treat or prevent a disease in a subject. A method may include administering to the subject a composition that includes the genome-edited primary NK cell described herein or produced by the method described herein. The disease could include, for example, cancer, a precancerous condition, infection with a pathogen (including, for example, malaria), or a viral infection. In some embodiments, it is preferred that the cells are used for cancer immunotherapy.

A genome-edited primary NK cell may be administered to a subject alone or in combination with one or more other therapies. For example, a genome-edited primary NK cell may be administered to a subject in combination a pharmaceutical composition that includes the active agent and a pharmaceutically acceptable carrier and/or in combination with a cellular therapy including, for example, a chimeric antigen receptor T cell (CAR-T). The NK cell may be administered to a patient, preferably a mammal, and more preferably a human, in an amount effective to produce the desired effect. The NK cell may be administered in a variety of routes, including, for example, intravenously, intratumorally, intraarterially, transdermally, via local delivery by catheter or stent, via a needle or other device for intratumoral injection, subcutaneously, etc. The NK cell may be administered once or multiple times. A physician having ordinary skill in the art may determine and prescribe the effective amount and dosing of an adaptive NK cell and, optionally, the pharmaceutical composition required.

The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CIVIL), Hodgkin's disease, and/or multiple myeloma. The cancer may be a metastatic cancer.

The virus may include, for example, a herpes virus, including for example, CMV, Varicella zoster virus (VZV), Epstein-Barr virus (EBV), a herpes simplex virus (HSV) or Kaposi's sarcoma-associated herpesvirus (KSHV); or a lentivirus, including for example, human immunodeficiency virus (HIV).

In a further aspect, a genome-edited primary NK cell may be administered to inhibit the growth of a tumor in a subject. In some embodiments, the tumor may include a solid tumor.

A genome-edited primary NK cell may be administered or prepared in a subject before, during, and/or after other treatments. Such combination therapy may involve administering a genome-edited primary NK cell before, during and/or after the use of other anti-cancer and/or anti-viral agents including, for example, a cytokine; a chemokine; a therapeutic antibody including, for example, a high affinity anti-CMV IgG antibody; an NK cell receptor ligand, including, for example, BiKE or TRiKE; an adjuvant; an antioxidant; a chemotherapeutic agent; and/or radiation. The administration or preparation may be separated in time from the administration of other anti-cancer agents and/or anti-viral agents by hours, days, or even weeks. Additionally or alternatively, the administration or preparation may be combined with other biologically active agents or modalities such as, but not limited to, an antineoplastic agent, and non-drug therapies, such as, but not limited to, surgery.

### ILLUSTRATIVE EMBODIMENTS

### Illustrative Embodiments of a Genome-Edited Primary NK Cell

1. A genome-edited primary natural killer (NK) cell.
2. The genome-edited primary NK cell of embodiment 1, wherein the NK cell comprises a cell expressing CD16.
3. The genome-edited primary NK cell of either of embodiments 1 or 2, wherein the NK cell comprises a cell expressing CD56.
4. The genome-edited primary NK cell of any one of embodiments 1 to 3, wherein the NK cell comprises a CD3⁻ cell.
5. The genome-edited primary NK cell of any one of embodiments 1 to 4, wherein the NK cell comprises a cell isolated from peripheral blood, umbilical cord cells, ascites, or a solid tumor.
6. The genome-edited primary NK cell of any one of embodiments 1 to 5, wherein the NK cell comprises a non-clonal cell.
7. The genome-edited primary NK cell of any one of embodiments 1 to 6, wherein the NK cell comprises a proliferating cell.
8. The genome-edited primary NK cell of any one of embodiments 1 to 7, wherein the NK cell comprises a stimulated NK cell.
9. The genome-edited primary NK cell of embodiment 8, wherein the stimulated NK cell comprises an NK cell exposed to a cytokine.
10. The genome-edited primary NK cell of embodiment 9, wherein the cytokine is bound to an artificial antigen presenting cell (aAPC).
11. The genome-edited primary NK cell of any one of embodiments 1 to 10, wherein the NK cell is a mammalian cell.
12. The genome-edited primary NK cell of any one of embodiments 1 to 11, wherein the NK cell is a human cell.
13. The genome-edited primary NK cell of any one of embodiments 1 to 12 wherein a gene is deleted.
14. The genome-edited primary NK cell of any one of embodiments 1 to 13, wherein a gene comprises a point mutation.
15. The genome-edited primary NK cell of any one of embodiments 1 to 14, wherein the cell has undergone homologous recombination.
16. The genome-edited primary NK cell of any one of embodiments 1 to 15, wherein the cell comprises an exogenous gene.
17. The genome-edited primary NK cell of embodiment 16, wherein the exogenous gene comprises a marker gene.
18. The genome-edited primary NK cell of embodiment 11 or embodiment 12, wherein the exogenous gene comprises nucleic acids encoding a chimeric antigen receptor.
19. The genome-edited primary NK cell of any one of embodiments 1 to 18, wherein the NK cell exhibits increased antibody-dependent cell cytotoxicity (ADCC) relative to a non-genome-edited primary NK cell.
20. The genome-edited primary NK cell of any one of embodiments 1 to 19, wherein the NK cell comprises a modification that alters expression or activity of CD16.
21. The genome-edited primary NK cell of any one of embodiments 1 to 20, wherein the NK cell comprises an ADAM17 cleavage-resistant CD16.
22. The genome-edited primary NK cell of any one of embodiments 1 to 21, wherein the NK cell comprises a modification that alters expression or activity of at least one of ADAM17, TIGIT, PD1, CISH, CCRS, NKG2A, and AAVS1.
23. The genome-edited primary NK cell of any one of embodiments 1 to 22, wherein the NK cell comprises a modification of a noncoding region of the genome.
24. The genome-edited primary NK cell of any one of embodiments 1 to 23, wherein the genome-edited primary NK cell comprises a modification in at least one of an activating receptor, an inhibitory receptor, an adaptor molecule, a downstream signaling molecule, a component of a cytotoxic granule, a cytokine, a chemokine, a cytokine receptor, and a chemokine receptor.
25. The genome-edited primary NK cell of any one of embodiments 1 to 24, wherein the genome-edited primary NK cell exhibits at least one of increased survival, increased capacity to kill cancer cells, and increased stimulation-induced cytokine production relative to a non-genome-edited primary NK cell.
26. The genome-edited primary NK cell of any one of embodiments 1 to 25, wherein the genome-edited primary NK cell exhibits increased expression of an activating receptor relative to a non-genome-edited primary NK cell.
27. The genome-edited primary NK cell of embodiment 26, wherein the activating receptor comprises at least one of CD16, IL-15 receptor, CD94-NKG2C, NKG2D, 2B4, DNAM-1 (CD226), a member of the KIR2DS family, a member of the KIR2DS Family, a member of the KIR3DS family, NKG2C, NKG2D, NKG2E, PILR (CD99), NKp30, NKp44, NKp46, NKp80, Sema4D (CD100), and CD160.
28. The genome-edited primary NK cell of any one of embodiments 1 to 27, wherein the genome-edited primary NK cell exhibits decreased expression of an inhibitory receptor relative to a non-genome-edited primary NK cell.
29. The genome-edited primary NK cell of embodiment 28, wherein the inhibitory receptor comprises at least one of PD-1, CD94-NKG2A, TIGIT, CISH, NKG2A, a member of the KIR2DL family, a member of the KIR3DL family, KLRG1, LILR. 2B4 (CD48), CD96 (Tactile), LAIR1, KLB1 (CD161), CEACAM-1, SIGLEC3, SIGLEC7, SIGLEC9, and CTLA4.
30. The genome-edited primary NK cell of any one of embodiments 1 to 29, wherein the genome-edited primary NK cell exhibits increased capacity to expand relative to a non-genome-edited primary NK cell.
31. The genome-edited primary NK cell of embodiment 30, wherein the genome-edited primary NK cell exhibits increased capacity to expand in vitro after co-culturing with at least one of a cytokine or a cancer cell line.
32. A method for treating or preventing a disease in a subject, the method comprising:
   administering to the subject a composition comprising the genome-edited primary NK cell of any one of embodiments 1 to 31.
33. The method of embodiment 32, wherein the disease comprises cancer, a precancerous condition, an infection with a pathogen, or a viral infection.

### Illustrative Embodiments of Methods of Editing a Genome of a Primary NK Cell

1. A method comprising editing a genome of a primary natural killer (NK) cell.
2. The method of embodiment 1, wherein the primary NK cell comprises a cell expressing CD16.
3. The method of either of embodiments 1 or 2, wherein the primary NK cell comprises a cell expressing CD56.
4. The method of any one of embodiments 1 to 3, wherein the primary NK cell comprises a CD3⁻ cell.
5. The method of any one of embodiments 1 to 4, wherein the primary NK cell comprises a cell isolated from peripheral blood, umbilical cord cells, ascites, or a solid tumor.
6. The method of any one of embodiments 1 to 5, wherein the primary NK cell comprises a non-clonal cell.
7. The method of any one of embodiments 1 to 6, wherein the primary NK cell comprises a proliferating cell.
8. The method of any one of embodiments 1 to 7, wherein the primary NK cell comprises a stimulated NK cell.
9. The method of embodiment 8, the method further comprising exposing a primary NK cell to a cytokine to produce a stimulated primary NK cell.
10. The method of embodiment 9, wherein the cytokine is bound to an artificial antigen presenting cell (aAPC).
11. The method of any one of embodiments 8 to 10, the method comprising treating the primary NK cell with a soluble cytokine.
12. The method of any one of embodiments 8 to 11, wherein the stimulated primary NK cell comprises an expanded NK cell.
13. The method of embodiment 12, wherein the stimulated primary NK cell comprises an NK cell in a population that has been expanded at least 5 fold.
14. The method of any one of embodiments 1 to 13, wherein the primary NK cell is a mammalian cell.
15. The method of any one of embodiments 1 to 14, wherein the primary NK cell is a human cell.
16. The method of any one of embodiments 1 to 15, the method comprising introduction of an exogenous protein or nucleic acid into the primary NK cell.
17. The genome-edited primary NK cell of embodiment 16 wherein the exogenous gene expresses a marker gene or a chimeric antigen receptor (CAR).
18. The method of any one of embodiments 1 to 17, the method comprising electroporation of the NK cell.
19. The method of embodiment 18, wherein electroporation comprises exposing the primary NK cell to at least 1700 volts, at least 1750 volts, at least 1800 volts, at least 1850 volts, at least 1900 volts, at least 1950 volts, at least 2000 volts, at least 2050 volts, at least 2100 volts, or at least 2150 volts.
20. The method of embodiment 18 or embodiment 19, wherein electroporation comprises exposing the primary NK cell to up to 1850 volts, up to 1900 volts, up to 1950 volts, up to 2000 volts, up to 2050 volts, up to 2100 volts, up to 2150 volts, up to 2200 volts, or up to 2250 volts.
21. The method of any one of embodiments 18 to 20, wherein electroporation comprises exposing the primary NK cell to at least 1 energy pulse, at least 2 energy pulses, at least 3 energy pulses, at least 4 energy pulses, or at least 5 energy pulses.
22. The method of any one of embodiments 18 to 21, wherein electroporation comprises exposing the primary NK cell to an energy pulse of last at least 2 milliseconds, at least 3 milliseconds, at least 4 milliseconds, at least 5 milliseconds, at least 7 milliseconds, at least 9 milliseconds, at least 10 milliseconds, at least 20 milliseconds, at least 30 milliseconds, or at least 40 milliseconds.
23. The method of any one of embodiments 1 to 22, the method comprising introducing a nuclease or a nucleic acid encoding a nuclease into the primary NK cell.
24. The method of embodiment 23, wherein the nuclease comprises Cas9.
25. The method of embodiment 23, wherein the nuclease comprises a transcription activator-like effector nuclease (TALEN) or a zinc-finger nuclease (ZFN).
26. The method of any one of embodiments 1 to 25, the method comprising introducing a guide RNA (gRNA).
27. The method of embodiment 26, where the gRNA comprises at least one of the gRNAs of Table 1B.
28. The method of embodiment 26 or embodiment 27, wherein the gRNA comprises a chemically modified gRNA.
29. The method of embodiment 28, wherein the chemically modified gRNA comprises 2′-O-methyl (M), 2′-O-methyl-3′-phosphorothioate (MS), or 2′-O-methyl-3′-thiophosphonoacetate (MSP).
30. The method of any one of embodiments 1 to 29 comprising introducing Natronobacterium gregoryi Argonaute (NgAgo) and a guide DNA(gDNA).
31. The method of any one of embodiments 1 to 30, wherein editing the genome comprises editing a gene for CD16.
32. The genome-edited primary NK cell of any one of embodiments 1 to 31, wherein the NK cell comprises a modification that alters expression or activity of at least one of ADAM17, TIGIT, PD1, CISH, CCR5, NKG2A, and AAVS1.
33. The method of any one of embodiments 1 to 32, wherein editing the genome comprises editing a gene for at least one of an activating receptor, an inhibitory receptor, an adaptor molecule, a downstream signaling molecule, a component of a cytotoxic granule, a cytokine, a chemokine, a cytokine receptor, and a chemokine receptor.
34. The method of embodiment 33, wherein the activating receptor comprises CD16, IL-15, IL-15 receptor, CD94-NKG2C, NKG2D, 2B4, DNAM-1 (CD226), a member of the KIR2DS family, a member of the KIR3DS family, NKG2C, NKG2D, NKG2E, PILR (CD99), NKp30, NKp44, NKp46, NKp80, Sema4D (CD100), or CD160.
35. The method of either of embodiments 33 or 34, wherein editing the gene for an activating receptor comprises introducing an activating mutation.
36. The method of any one of embodiments 33 to 35, wherein editing the gene for an activating receptor comprises upregulating expression of the gene.
37. The method of any one of embodiments 1 to 36, wherein editing the genome comprises editing a gene for an inhibitory receptor.
38. The method of embodiment 37, wherein the inhibitory receptor comprises PD-1, CD94-NKG2A, TIGIT, CISH, NKG2A, a member of the KIR2DL family, a member of the KIR3DL family, KLRG1, LILR. 2B4 (CD48), CD96 (Tactile), LAIR1, KLB1 (CD161), CEACAM-1, SIGLEC3, SIGLEC7, SIGLEC9, or CTLA4.
39. The method of either of embodiments 37 or 38, wherein editing the gene for an inhibitory receptor comprises introducing an inactivating mutation.
40. The method of any one of embodiments 1 to 39, wherein editing the genome comprises editing a gene for an adaptor molecule.
41. The method of embodiment 40, wherein the adaptor molecule comprises EAT2, DAP10, DAP12, or CD3zeta.
42. The method of any one of embodiments 1 to 41, wherein editing the genome comprises editing a gene for a disintegrin and metalloprotease-17 (ADAM17).
43. The method of any one of embodiments 1 to 42, wherein editing the genome comprises editing a ADAM17 cleavage region of CD16.
44. The method of any one of embodiments 1 to 43, wherein editing the genome comprises editing a noncoding region of the genome.
45. The method of any one of embodiments 1 to 44, wherein the method further comprises selecting an NK cell.
46. The method of embodiment 45, wherein the selection is performed after editing the genome.

47. The method of either of embodiments 45 or 46, wherein the NK cell is selected for the an edited genome.
48. The method of any one of embodiments 1 to 47, wherein the method further comprises expanding an NK cell.
49. The method of embodiment 48, wherein expanding an NK cell comprises incubating the NK cell with an artificial antigen-presenting cell.
50. The method of either of embodiments 48 or 49, wherein expanding an NK cell comprises incubating the NK cell with a cell expressing membrane-bound IL-21 or an aAPC bound to IL-21.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Medias

### Culturing Media:

B0 Media
   300 milliliters (mL) DMEM
   150 mL Ham F12
   50 mL Human Serum (10%)
   100 Units per milliliter (U/mL) Pen; 100 μg/mL Strep
   2-mercaptoethanol (20 micromolar (μM))
   Ethanolamine (50 μM)
   Ascorbic Acid (10 micrograms per milliliter (μg/mL))
   Sodium Selenite (1.6 nanograms per milliliter (ng/mL))
NK92 Media
   500 mL Alpha MEM with nucleosides
   62.5 mL Horse Serum
   62.5 mL Human Serum (10%)
   100 U/mL Pen; 100 μg/mL Strep
   2-mercaptoethanol (0.1 mM)
   500 U/mL IL-2

### Freezing Media:

45 mL FBS (heat inactivated) and 5 mL DMSO or
CRYOSTOR Cell Preservation Media 10 (StemCell Technologies, Vancouver, BC, Canada)

### Materials

TABLE 1
Guide RNA target sequences:

| | | |
|---|---|---|
| CCR5 gRNA | GGCAGCATAGTGAGCCCAGA | SEQ ID NO: 1 |
| PD1 gRNA | CCTGCTCGTGGTGACCGAAG | SEQ ID NO: 2 |
| ADAM17 gRNA | GAACCACGCTGGTCAGGAAT | SEQ ID NO: 3 |
| TIGIT gRNA | GACCTGGGTCACTTGTGCCG | SEQ ID NO: 4 |
| CISH gRNA | GGGTTCCATTACGGCCAGCG | SEQ ID NO: 5 |
| NKG2A gRNA | AACAACTATCGTTACCACAG | SEQ ID NO: 6 |
| CD16 gRNA #2 | TCTCATCATTCTTTCCACCT | SEQ ID NO: 7 |
| CD16 gRNA #3 | AGGTGGAAAGAATGATGAGA | SEQ ID NO: 8 |
| AAVS1 | GTCACCAATCCTGTCCCTAG | SEQ ID NO: 9 |

TABLE 1B
Guide RNA sequences

| | | |
|---|---|---|
| CCR5 gRNA | GGCAGCAUAGUGAGCCCAGA | SEQ ID NO: 10 |
| PD1 gRNA | CCUGCUCGUGGUGACCGAAG | SEQ ID NO: 11 |
| ADAM17 gRNA | GAACCACGCUGGUCAGGAAU | SEQ ID NO: 12 |
| TIGIT gRNA | GACCUGGGUCACUUGUGCCG | SEQ ID NO: 13 |
| CISH gRNA | GGGUUCCAUUACGGCCAGCG | SEQ ID NO: 14 |
| NKG2A gRNA | AACAACUAUCGUUACCACAG | SEQ ID NO: 15 |
| CD16 gRNA #2 | UCUCAUCAUUCUUUCCACCU | SEQ ID NO: 16 |
| CD16 gRNA #3 | AGGUGGAAAGAAUGAUGAGA | SEQ ID NO: 17 |
| AAVS1 | GUCACCAAUCCUGUCCCUAG | SEQ ID NO: 18 |

TABLE 2
Primer sequences:

| Target | Forward primer |
|---|---|
| CCR5 | GCACAGGGTGGAACAAGATGG (SEQ ID NO: 19) |
| PD1 | GGGTGAAGGCTCTTAGTAGG (SEQ ID NO: 21) |
| ADAM17 | CCCGATGTGAGCAGTTTTCC (SEQ ID NO: 23) |
| TIGIT | GAGGAGCAACAGGATGGACT (SEQ ID NO: 25) |
| CISH | CTTCTGCGTACAAAGGGCTG (SEQ ID NO: 27) |
| NKG2A | CAATGGGAGATGAGGGTTTG (SEQ ID NO: 29) |
| CD16 | CCCCACCATTCCTACCACTT (SEQ ID NO: 31) |
| AAVS1 | ACTCCTTTCATTTGGGCAGC (SEQ ID NO: 33) |
| Target | Reverse primer |
| CCR5 | CACCACCCCAAAGGTGACCGT (SEQ ID NO: 20) |
| PD1 | CAGGCTCTCTTTGATCTGC (SEQ ID NO: 22) |
| ADAM17 | GAGACAGGCCCATCTCCTTT (SEQ ID NO: 24) |
| TIGIT | TGCAGTGTTTCAGGATTGCA (SEQ ID NO: 26) |
| CISH | GAGAGTCTGATGGGAGAGGC (SEQ ID NO: 28) |
| NKG2A | CAATGAGAACTCTATTCCCTGAAA (SEQ ID NO: 30) |
| CD16 | CCCCACCATTCCTACCACTT (SEQ ID NO: 32) |
| AAVS1 | GGTTCTGGCAAGGAGAGAGA (SEQ ID NO 34) |

### Methods

### Design and Construction of Guide RNAs

Guide RNAs (gRNAs) were designed to the desired region of a gene using the CRISPR Design Program (Zhang Lab, MIT 2015, available on the world wide web at crispr.mit.edu). Multiple gRNAs were chosen based on the highest ranked values determined by off-target locations. The gRNAs were ordered in oligonucleotide pairs: 5′-CACCG-gRNA sequence-3′ and 5′-AAAC-reverse complement gRNA sequence-C-3′. The gRNAs were cloned together using a modified version of the target sequence cloning protocol (Zhang Lab, MIT, available on the world wide web at crispr.mit.edu). The oligonucleotide pairs were phosphorylated and annealed together using T4 PNK (New England Biolabs, Ipswich, Mass.) and 10X T4 Ligation Buffer (New England Biolabs, Ipswich, Mass.) in a thermocycler with the following protocol: 37° C. 30 minutes, 95° C. 5 minutes, and then ramped down to 25° C. at 5° C./minute. pENTR1 vector digested with FastDigest BbsI (Fermentas, Thermo Fisher Scientific, Waltham, Mass.), FastAP (Fermentas, Thermo Fisher Scientific, Waltham, Mass.) and 10X Fast Digest Buffer are used for the ligation reaction. The digest pENTR1 vector was ligated together with the phosphorylated and annealed oligo duplex (dilution 1:200) from the previous step using T4 DNA Ligase and Buffer (NEB). The ligation was incubated at room temperature for at least 1 hour and then transformed and mini-prepped (GeneJET Plasmid Miniprep Kit, Life Technologies, Carlsbad, Calif.). The plasmids were sequenced to confirm the proper insertion.

Validation of gRNAs

293T cells were plated out at a density of 1×10⁵ cells per well in a 24 well plate. 150 microliters (μL) of Opti-MEM medium was combined with 1.5 μg of gRNA plasmid, 1.5 micrograms (μg) of Cas9 plasmid, and 100 ng of GFP. Another 150 μL of Opti-MEM medium was combined with 5 μL of Lipofectamine 2000 Transfection reagent (Invitrogen, Carlsbad, Calif.; Life Technologies, Carlsbad, Calif.). The solutions were combined together and incubated for 10 to 15 minutes at room temperature. The DNA-lipid complex was added dropwise to one well of the 24 well plate. Cells were incubated for 3 days at 37° C. and then genomic DNA was collected using the GeneJET Genomic DNA Purification Kit (Thermo Fisher Scientific, Waltham, Mass.).

For FIGS. 1-7 (as appropriate), activity of the gRNAs was quantified by a Surveyor Digest, gel electrophoresis, and densitometry (Guschin et al., Methods Mol. Biol., 2010, 649:247-256).

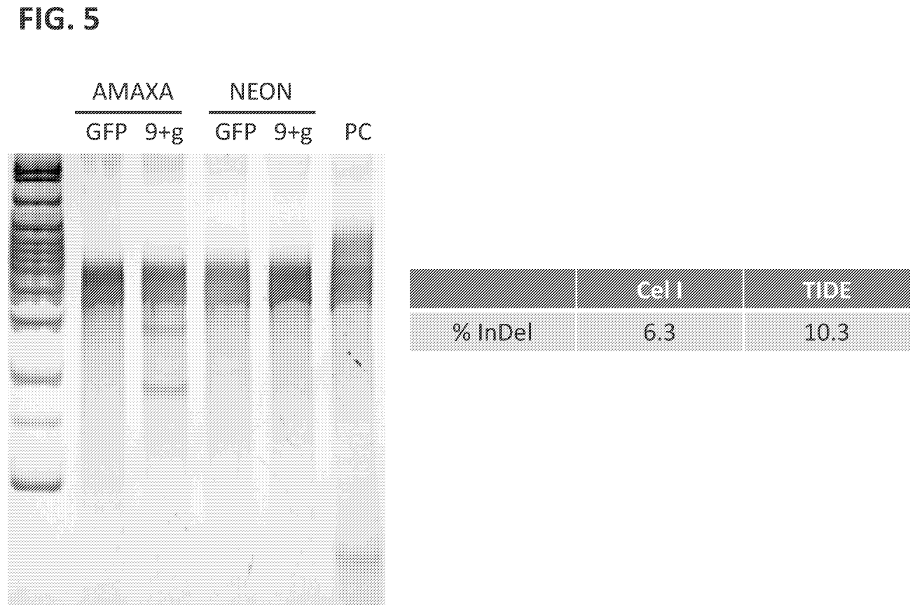

Additionally or alternatively (including for FIGS. 5 and 8-16, as appropriate), activity of the gRNAs was quantified by the Tracking of Indels by Decomposition (TIDE) algorithm (available on the world wide web at tide-calculator.nki.nl/). Briefly, the edited region was amplified by PCR using region-specific primers, and sent to ACGT, Inc. (Wheeling, Ill.) for Sanger Sequencing. Chromatogram files returned from ACGT, Inc. were uploaded to the TIDE website for analysis of editing efficiency.

Isolation of Peripheral Blood Mononuclear Cells (PBMCs) from a Leukopak or TrimaCone

The blood cells were diluted 3:1 with chilled 1X PBS. The diluted blood was added dropwise (very slowly) over 15 mL of Lymphoprep (Stem Cell Technologies, Vancouver, Canada) in a 50 mL conical. Cells were spun at 400×g for 25 minutes with no brake. The buffy coat was removed and placed into a new conical. The cells were washed with chilled 1X PBS and spun for 400×g for 10 minutes (with brake). The supernatant was removed, cells were resuspended in media and counted. Cells were either frozen as PBMCs or used immediately to purify NK cells.

Isolation of CD3⁻CD56⁺ NK Cells

PBMCs were thawed, if necessary, collected via density gradient centrifugation, and counted; the cell density was adjusted to 5×10⁷ cells/mL and transferred to a 14 mL polystyrene round-bottom tube. Using the EASYSEP Human NK cell Enrichment Kit (Stem Cell Technologies, Vancouver, Canada), 50 μL/mL of the Isolation Cocktail was added to the cells. The mixture was mixed by pipetting and then incubated for 15 minutes at room temperature. After the incubation, the RAPIDSPHERES were vortexed for 30 seconds and then added (100 μL/mL) to the sample; mixed by pipetting up and down and incubated for 5 minutes at room temperature. The mixture was topped off to 5 mL for samples less than 2 mL (<10⁸ cells) or topped off to 10 mL for samples more than 2 mL. The polystyrene tube was added to the “Big Easy” magnet; incubated at room temperature for 2.5 minutes. The magnet and tube, in one continuous motion, were inverted, pouring off the enriched cell suspension into a new tube.

### Culturing of the NK92 Cell Line

The NK92 cell line was cultured in the NK92 Media, as described above, at a concentration between 1×10⁵ cells/mL and 9×10⁵ cells/mL. Cell clumps were disrupted every 2-3 days by pipetting. When necessary, cells were split into new media at a concentration of 1×10⁵ cells/mL.

Stimulation of CD3⁻CD56⁺NK Cells

### Activation:

CD3⁻CD56⁺ NK cells were counted and plated out at a density of 1×10⁶ cells/mL in a 24 well plate. CD335/CD2 biotin-labeled antibodies from a Human NK Cell Expansion Activator Kit (Miltenyi Biotech, Bergisch Gladbach, Germany) were added 2:1 (beads:cells) to the cells after being loaded onto the MACSiBead Particles according to manufacturer's instructions. IL-2 (Peprotech, Rocky Hill, N.J.) was added at a concentration of 500 IU/mL. Some cells were also cultured with 10 ng/ml of IL-15 (Peprotech, Rocky Hill, N.J.) in addition to IL-2. Cells were incubated for 7 days, counted, and subjected electroporation or nucelofection.

### Proliferation:

1) Membrane-bound IL-21: PBMC or CD3-CD56+ NK cells were counted and plated out at a density of 1.25×10⁵ cells/mL or 2.5×10⁵ cells/mL and co-cultured with artificial antigen presenting cells (aAPC; clone 9; Denman et al., PLoS One, 2012, 7(1): e30264 doi: 10.1371/journal.pone.0030264) at a 2:1 (feeder:PBMC/NK) ratio in either a 24-well plate or T-75 flask. Prior to co-culture the aAPC were irradiated at 100 or 200 Gray with an X-Ray irradiator. PBMC/NK and feeder cells were suspended in B0 media containing 50 U/mL of IL-2 (Peprotech, Rocky Hill, N.J.). On days 3 and 5, cells were collected and spun down. Half of the media was replaced with fresh B0 media containing 50 U/mL IL-2. On day 7, cells were counted and re-cultured with aAPC cells at a 1:1 ratio in fresh BO media containing 50 U/mL IL-2. On days 10 and 12, half the media was replaced as above. On day 14, CD3^{-CD}56⁺ NK cells were purified from PBMC expanded cells and frozen down. If the starting cells were CD3⁻CD56⁺ NK cells, the cells were re-cultured with irradiated aAPC at a 1:1 ratio.

2) Soluble IL-21: CD3⁻CD56⁺ NK cells were counted and plated out at a density of 1.25×10⁵ cells/mL and co-cultured with K562 cells (ATCC) at a 2:1 (feeder:NK) ratio in a 24-well plate. Prior to co-culture, the K562 cells were irradiated at 200 Gray with an X-Ray irradiator. The NK and feeder cells were suspended in BO media containing 50 U/ml of IL-2 (Peprotech, Rocky Hill, N.J.). Different concentrations of soluble IL-21 (Peprotech, Rocky Hill, N.J.) were added to the culture. On days 3 and 5, cells were collected and spun down. Half of the media was replaced with fresh BO media containing 50 U/mL IL-2. On day 7, cells were counted and re-cultured with irradiated K562 cells at a 1:1 ratio in fresh BO media containing 50 U/mL IL-2. On days 10 and 12, half of the media was replaced as above.

3) Cells were expanded using the CellXVivo Human NK Cell Expansion Kit (R&D Systems, Minneapolis, Minn.). Twenty five million PBMC were cultured in a T-75 flask that had been coated the night before with 6 mL of NK Cell Expander 1. The PBMCs were cultured in 25 mL of expansion media containing NK Cell Expanders 2, 3, 4 and 5 in Human NK Cell Expansion Media. On Day 4, cells were harvested, refreshed with 25 mL of NK Cell Expansion Media (containing NK Cell Expanders 2-5) and returned to the original flask. On Day 7, cells were collected, spun down and evenly distributed to 4 new flasks that were coated as before with Expander 1. The cells in the new flasks were culured in 25 mL of Expansion media (containing NK Cell Expanders 2-5). On Day 10, Expansion Media was refreshed as described on Day 4. The expanded NK cells were collected for electroporation on Day 14 post-culture.

AMAXA Nucleofection of CD3⁻CD56⁺ NK Cells

Unstimulated NK cells or stimulated NK cells (for example, NK cells subjected to activation and/or proliferation) were nucleofected using the AMAXA Human Macrophage Cell Nucleofector Kit (Lonza Group, Basel, Switzerland). Cells were counted and resuspended at of density of between 1×10⁶ and 3×10⁶ cells in per 100 μL of room temperature AMAXA buffer. 10 μg of GFP mRNA or 2.5 μg transposase/7.5 μg transposon of plasmids were added to the cell mixture. Cells were nucleofected using the Y-010 program. After nucleofection, cells were plated in 2 mL culturing media in a 12 well plate.

NEON Electroporation of CD3^{-CD}56⁺ NK Cells Using a T Cell Protocol

Unstimulated NK cells or stimulated NK cells electroporated using a T cell protocol were resuspended at a density of 30×10⁶ cells per 1 mL of room temperature Buffer T (NEON Transfection Kit, Invitrogen, Carlsbad, Calif.); 10 μg of GFP mRNA or 2.5 μg transposase/7.5 μg transposon of plasmids were added to the cell mixture; cells were electroporated with 3 pulses of 1400 volts of 10 milliseconds using a NEON platform (Invitrogen, Carlsbad, Calif.).

### Flow Cytometry

Unless otherwise indicated, transfected, electroporated, or nucleofected NK cells were analyzed by flow cytometry 48 hours to 72 hours after transfection, electroporation, or nucleofection for expression of GFP. Cells were prepped by washing with chilled 1X PBS with 0.5% FBS and stained with BV411 anti-human CD3E (eBiosciences, San Diego, Calif.), APC anti-human CD56 (Miltenyi Biotec, Bergisch Gladbach, Germany), and Fixable Viability Dye eFlour 780 (eBiosciences, San Diego, Calif.) or Sytox Blue. Cells were analyzed using a LSR II (BD Biosciences, San Jose, Calif.) and FlowJo v.9.

Gene Knockout in CD3⁻CD56⁺ NK Cells

Unstimulated or stimulated CD3⁻CD56⁺ NK cells were nucleofected using the AMAXA nucleofection system (Human Macrophage Kit, Lonza Group, Basel, Switzerland). Cells were counted and resuspended at a density of 3.0×10⁶ cells in 100 μL of Resuspension buffer, and the following reagents were added to the cells and buffer prior to nucleofection: 10 μg, 15 μg, or 20 μg mRNA Cas9 (TriLink. BioTechnologies, San Diego, Calif.), and 10 μg, 20 μg, or 30 μg gRNA (TriLink BioTechnologies, San Diego, Calif.). Three days after nucleofection, cells were harvested and gRNA activity was measured as above.

Alternatively, unstimulated or stimulated CD3⁻CD56⁺ NK cells were transfected using the NEON Transfection Kit and System (Invitrogen, Carlsbad, Calif.). Cells were counted and resuspended at a density of 30×10⁶ cells per 1 mL of room temperature Buffer T (NEON Transfection Kit, Invitrogen, Carlsbad, Calif.), and the following reagents were added to the cells and buffer prior to electroporation: 15 μg Cas9 mRNA (TriLink BioTechnologies, San Diego, Calif.), and 15 μg gRNA (TriLink BioTechnologies, San Diego, Calif.). Cells were electroporated with 2 pulses of 1850 volts of 10 milliseconds.

After electroporation, cells were plated in 2 mL B0 medium supplemented with 1 ng/mL IL-15. This reaction was scaled down for 10 μL reactions (3×10⁵ cells) by using 1/10 the amount of reagents listed above and in this case cells were plated in 200 μL or 500 μL B0 medium supplemented with 1 ng/mL IL-15. Medium was replaced every other day after electroporation. Five days after electroporation, cells were harvested and gRNA activity was measured by TIDE analysis as described above.

Homologous Recombination in CD3⁻CD56⁺ NK Cells

Stimulated CD3⁻CD56⁺ NK cells were transfected using the NEON Transfection Kit and System (Invitrogen, Carlsbad, Calif.). Cells were counted and resuspended at a density of 30×10⁶ cells per 1 mL of room temperature Buffer T (NEON Transfection Kit, Invitrogen, Carlsbad, Calif.), and the following reagents were added to the cells prior to electroporation: 15 μg Cas9 mRNA (TriLink BioTechnologies, San Diego, CA), 15 μM gRNA, 1 μg or 10 μg of homologous recombination (HR) targeting vector that expresses GFP were used for to examine HR. 1 μg or 10 μg of HR targeting vector alone or 15 μg Cas9 with 15 μg gRNA were used as controls. Five days after electroporation, NK cells were co-cultured with aAPC (1:1 ratio) for 7 days. To monitor for HR, cells were analyzed by flow cytometry and tested by PCR. For flow cytometry, cells were analyzed 5 days and 12 days post-electroporation to determine GFP expression and viability. Cells were analyzed using a LSR II (BD Biosciences, San Jose) and FlowJo v.9. To test for HR by PCR, gDNA was isolated from NK cells and amplified by PCR using accuprime taq DNA polymerase, high fidelity (Thermo Fisher Scientific Inc., Waltham Mass.). Primers were designed to amplify from within the targeting vector to and from AAVS to look for proper homologous recombination.

Transposon/Transposase-Mediated Chimeric Antigen Receptor integration into CD3⁻CD56⁺ NK Cells

Unstimulated CD3⁻CD56⁺ NK cells were nucleofected using the AMAXA nucleofection system (Human Macrophage Kit, Lonza Group, Basel, Switzerland). Cells were counted and resuspended at a density of 3.0×10⁶ cells in 100 of Nucleofector Solution, 2.5 μg Sleeping Beauty or PiggyBac Transposase and 7.5 μg of transposon vector containing the chimeric antigen receptor (CAR). As a control, NK cells were electroporated with only 2.5 μg of transposase.

Alternatively, unstimulated CD3^{-CD}56⁺ NK cells were transfected using the NEON Transfection Kit and System (Invitrogen, Carlsbad, Calif.), Cells were counted and resuspended at a density of 30×10⁶ cells per 1 mL of room temperature Buffer T (NEON Transfection Kit, Invitrogen, Carlsbad, Calif.), and the following reagents were added to the cells prior to electroporation: 2.5 μg Sleeping Beauty or PiggyBac Transposase and 7.5 μg of transposon vector containing the chimeric antigen receptor (CAR). As a control, NK cells were electroporated with only 2.5 μg of transposase. Cells were electroporated, cultured, and analyzed as described above.

### Results

To test the efficiency and toxicity of mRNA delivery to primary human NK cells, unstimulated NK cells were electroporated with in vitro transcribed mRNA encoding EGFP (TriLink Biotechnologies, San Diego, Calif.) using the AMAXA platform. Gene delivery rates as high as 85.7% with a viability of up to 86,2% were obtained across multiple donors (FIG. 1).

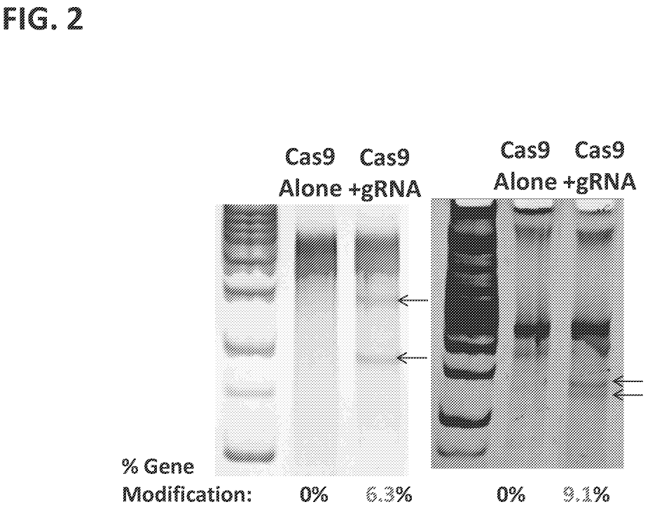

Using chemically modified gRNAs targeting CCRS (Target Sequence: GGC AGC ATA GTG AGC CCA GA (SEQ ID NO:1)) and PD1 (Target Sequence: CCT GCT CGT GGT GAC CGA AG (SEQ ID NO:2)) and Cas9 mRNA, gene modification of NK cells was induced (FIG. 2).

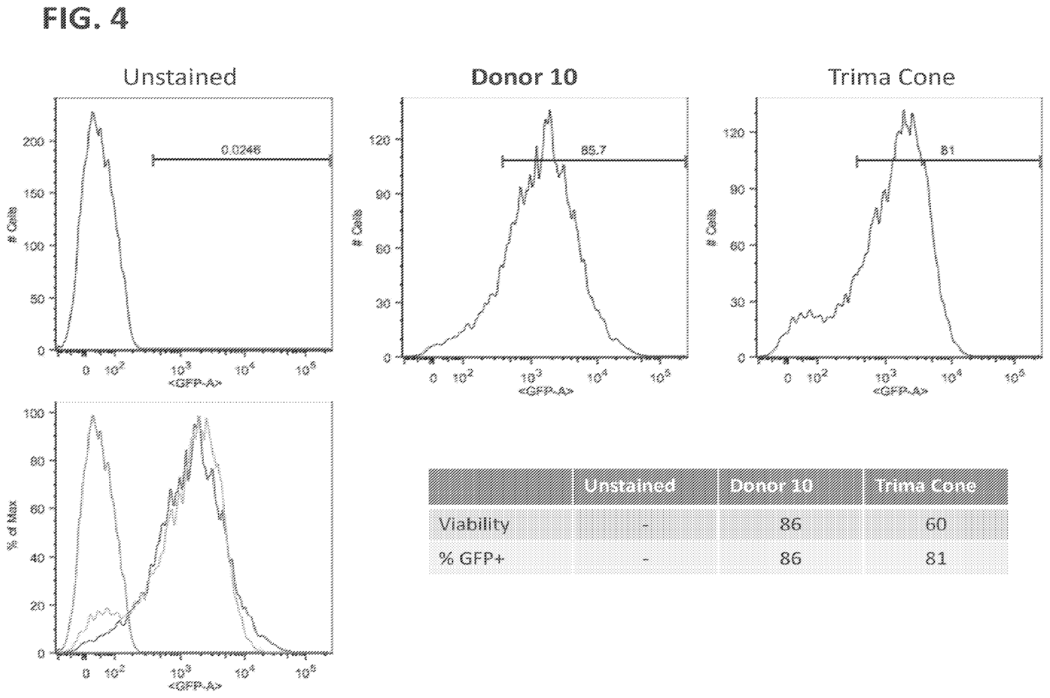

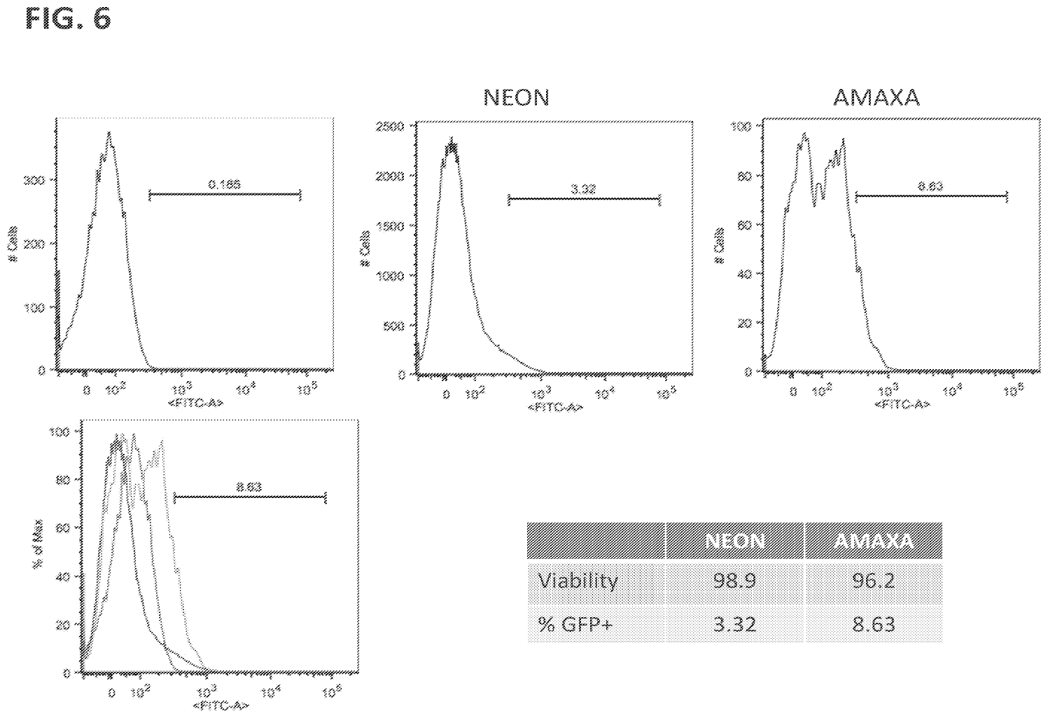

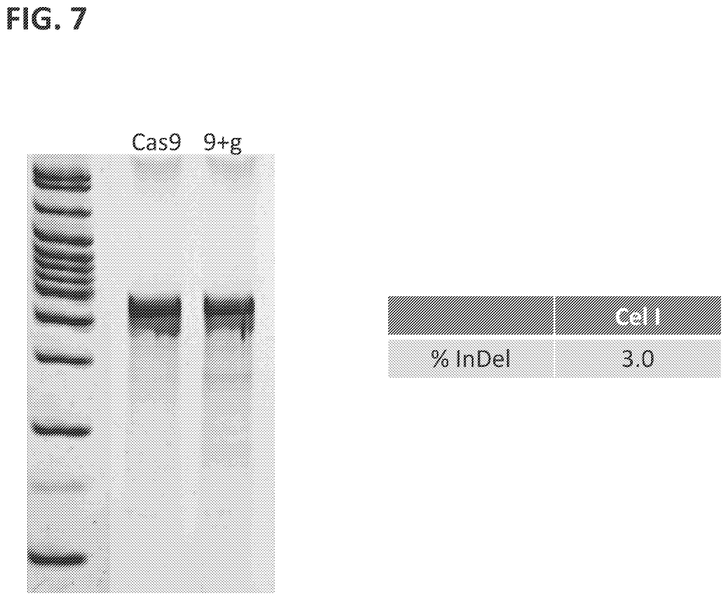

To compare transfection efficiency of the NEON (T cell protocol) and AMAXA platform (Macrophage Kit), 3 million unstimulated NK cells were electroporated with in vitro transcribed mRNA encoding EGFP. For unstimulated NK cells, the AMAXA system was almost 10 times as efficient as delivering EGFP mRNA compared to the NEON platform (FIG. 3). NK cells isolated from either frozen or freshly collected PBMC were transfected at equal efficiency using the AMAXA platform (FIG. 4). Unstimulated primary NK cells are amenable to gene editing using Cas9 and gRNA targeting CCR5 when using the AMAXA but not the NEON platform (FIG. 5). Unexpectedly, using these protocols, NK cells expanded using the cell line containing membrane-bound IL-21 were less efficiently transfected with EGFP mRNA (FIG. 6) and less amenable to editing (FIG. 7) using Cas9 mRNA and chemically modified gRNA than resting NK cells.

### Example 2

Unless otherwise indicated, the materials and methods of Example 1 were used.

### Methods

Optimized Electroporation of CD3⁻CD56⁺ NK Cells Using the NEON Transfection System

Unstimulated NK cells or stimulated NK cells (for example, NK cells subjected to activation and/or proliferation) were transfected using the NEON Transfection Kit and System (Invitrogen, Carlsbad, Calif.). Cells were counted and resuspended at a density of 30×10⁶ cells per 1 mL of room temperature Buffer T (NEON Transfection Kit, Invitrogen, Carlsbad, Calif.). To determine the optimal electroporation conditions, 3×10⁵ cells were electroporated with 1 μg of EGFP in 10 μL tips under a variety of conditions (see, for example, Table 3). Following electroporation, NK cells were cultured in 500 μL B0 supplemented with 1 ng/ml IL-15. Cell counts, viability and EGFP expression were measured 48 hours later.

Electroporation conditions applying less than 1700 volts were found to result in poor EGFP expression. Electroporation conditions applying more than 2000 volts were found to results in high levels of cell death.

The electroporation condition that resulted in the best cell count (that is, the highest survival following electroporation) and EGFP expression was determined (FIG. 8), and these optimized

NEON electroporation conditions (2 pulses of 1850 volts over 10 milliseconds) were used for all subsequent experiments. In subsequent experiments that used 3×10⁶ cells in a 100 μL tip, the NK cells were cultured following electroporation in 2 ml BO media containing 1 ng/ml IL-15 in a 12-well plate.

TABLE 3

| Sample | | Pulse | # of | Average live | Average |
|---|---|---|---|---|---|
| number | Voltage | width | pulses | (cells/mL) | viability |
| 3 | 1700 | 10 | 1 | | |
| 4 | 1700 | 10 | 2 | | |
| *5 | 1700 | 10 | 3 | 9.04E + 05 | 87.30% |
| 6 | 1750 | 20 | 2 | | |
| 7 | 1750 | 20 | 3 | | |
| 8 | 1700 | 30 | 1 | | |
| 9 | 1750 | 10 | 1 | | |
| *10 | 1750 | 10 | 2 | 7.77E + 05 | 88.05% |
| *11 | 1750 | 10 | 3 | 5.87E + 05 | 89.27% |
| 12 | 1850 | 10 | 1 | | |
| *13 | 1850 | 10 | 2 | 8.33E + 05 | 92.25% |
| *14 | 1850 | 10 | 3 | 5.31E + 05 | 81.57% |
| 15 | 1800 | 20 | 2 | | |
| 16 | 1800 | 20 | 3 | | |
| 17 | 1800 | 30 | 1 | | |
| 18 | 1850 | 20 | 1 | | |
| 19 | 1850 | 20 | 2 | | |
| 20 | 1850 | 20 | 3 | | |
| *21 | 1700 | 20 | 2 | 3.99E + 05 | 82.33% |
| *22 | 1700 | 20 | 3 | 2.99E + 05 | 75.51% |
| *23 | 1800 | 10 | 1 | 1.00E + 06 | 91.21% |
| *24 | 1800 | 10 | 2 | 5.93E + 05 | 90.60% |

An asterisk (*) next to sample number indicates good GFP/viability, as measured by flow cytometry

### Example 2A

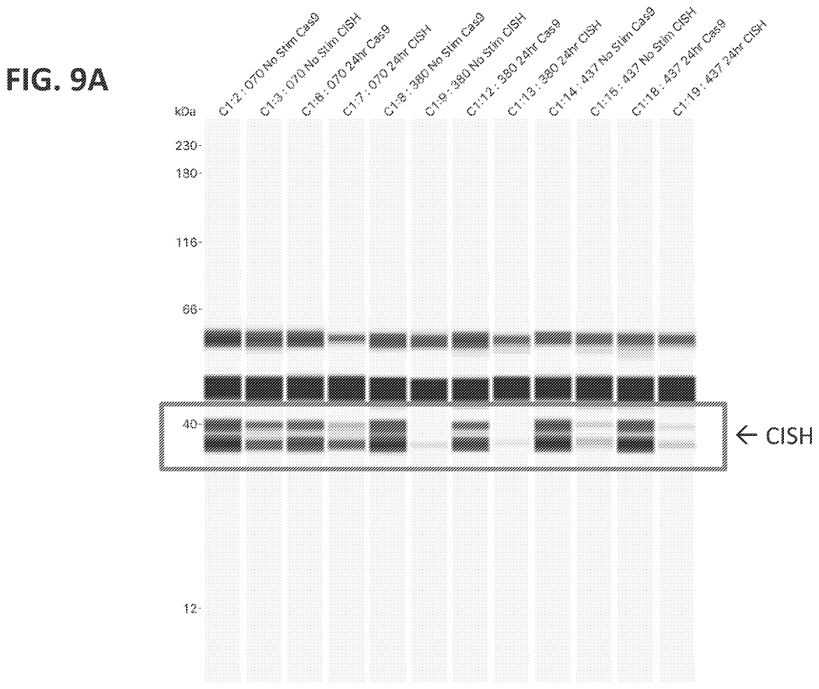

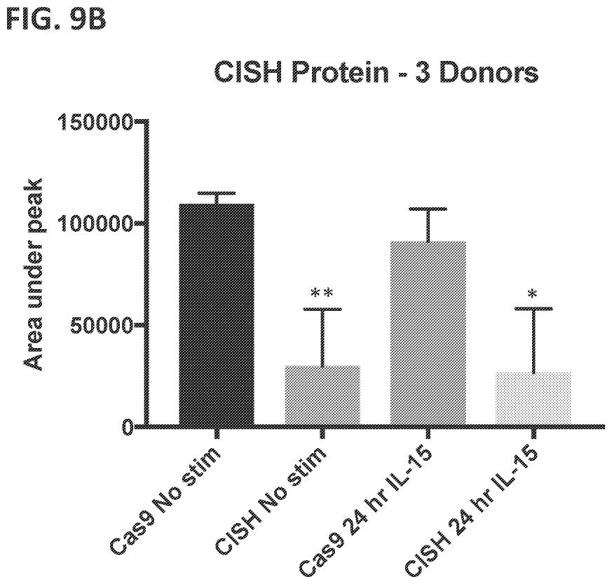

NK cells (3×10⁶) from three donors (donors 070, 380, and 437) stimulated with aAPCs were electroporated using the optimized NEON electroporation conditions with 10 μg Cas9 mRNA and 10 μg CISH gRNA. Five days after electroporation, cells were harvested and protein lysates were run with a CISH-specific antibody on a Wes machine (Protein Simple, San Jose, Calif.) and CISH expression (area under peak) normalized to (3-actin was analyzed using Compass Software (Protein Simple, Bio-Techne, Minneapolis, Minn.). Results are shown in FIG. 9.

### Example 2B

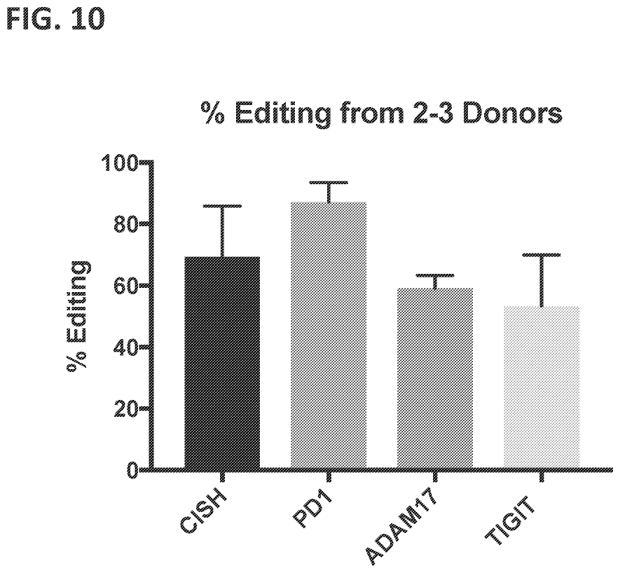

NK cells (3×10⁶) from three donors (donors 070, 380, and 437 for CISH and PD1) or from two donors (donors 407 and 613 for ADAM17 and TIGIT) stimulated with aAPCs were electroporated using the optimized NEON electroporation conditions with 10 μg Cas9 mRNA and 10 μg gRNA. Five days after electroporation, cells were harvested and genomic DNA was PCR-amplified at the target region. PCR products were sent to ACGT, Inc. (Wheeling, Ill.) for Sanger Sequencing and chromatograms were uploaded to the TIDE website for analysis of editing. Percent editing was calculated by comparing edited samples to matched donor samples electroporated with Cas9 mRNA alone. Results are shown in FIG. 10.

### Example 2C

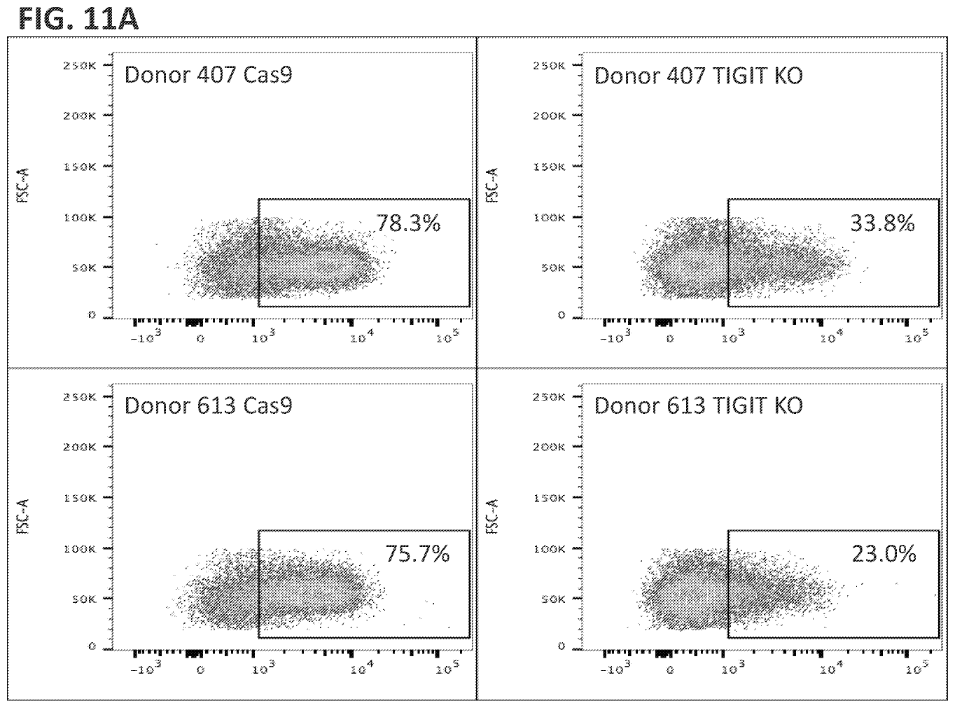

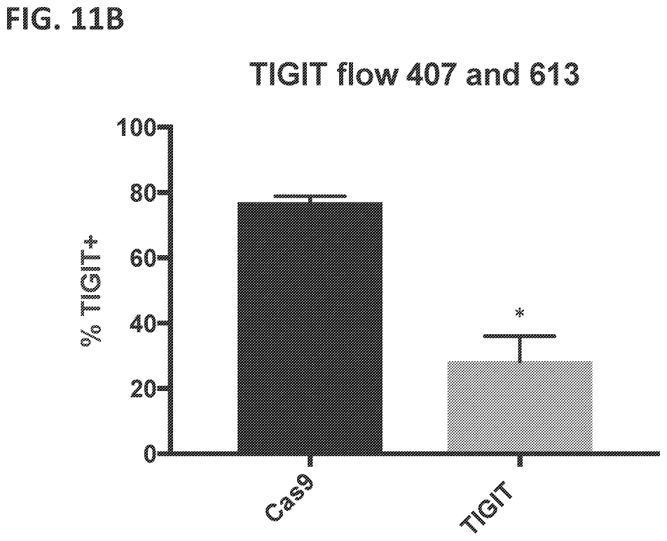

3×10⁶ NK cells from two donors (donors 407 and 613) stimulated with aAPCs were electroporated using the optimized NEON electroporation conditions with 10 μg Cas9 mRNA and 10 μg gRNA. Five days after electroporation, cells were harvested and stained with antibodies for CD56, CD3, TIGIT, and SYTOX Blue viability dye. Cells were gated on single cells (FSC-H vs FSC-A), CD56⁺, CD3⁻, SYTOX Blue⁻, and TIGIT⁺. TIGIT expression in knockout cells was compared to matched donor samples electroporated with Cas9 mRNA alone. Results are shown in FIG. 11.

### Example 2D

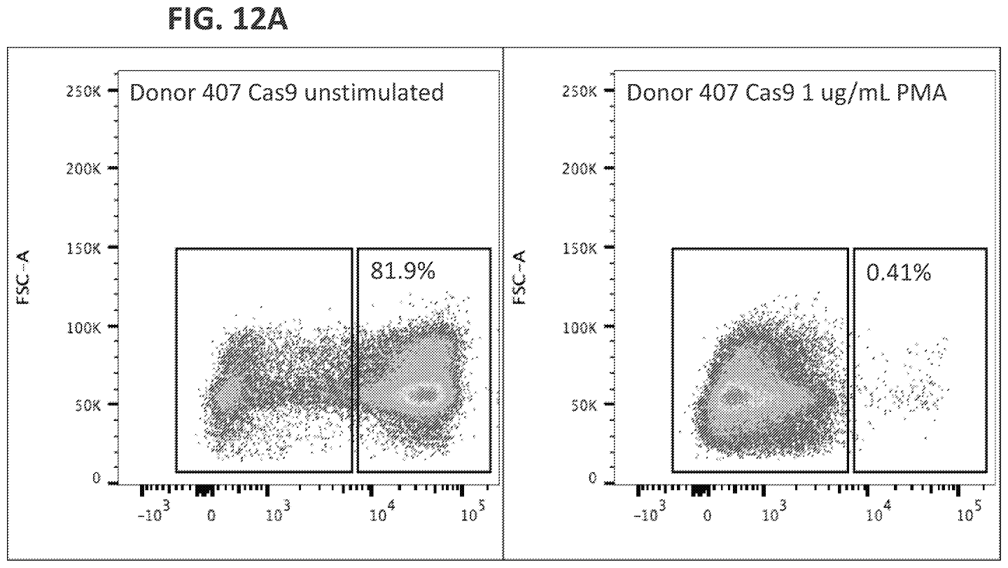

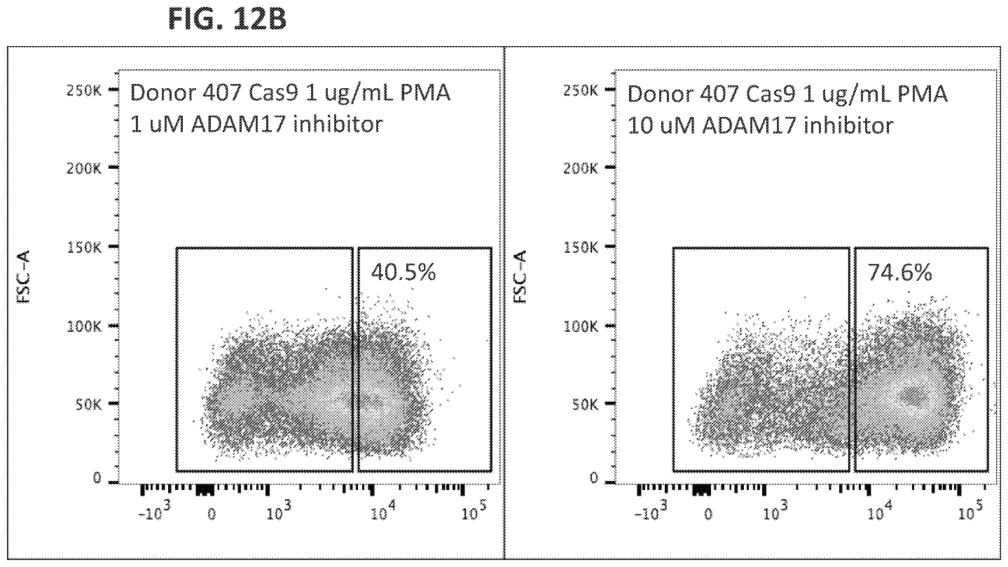

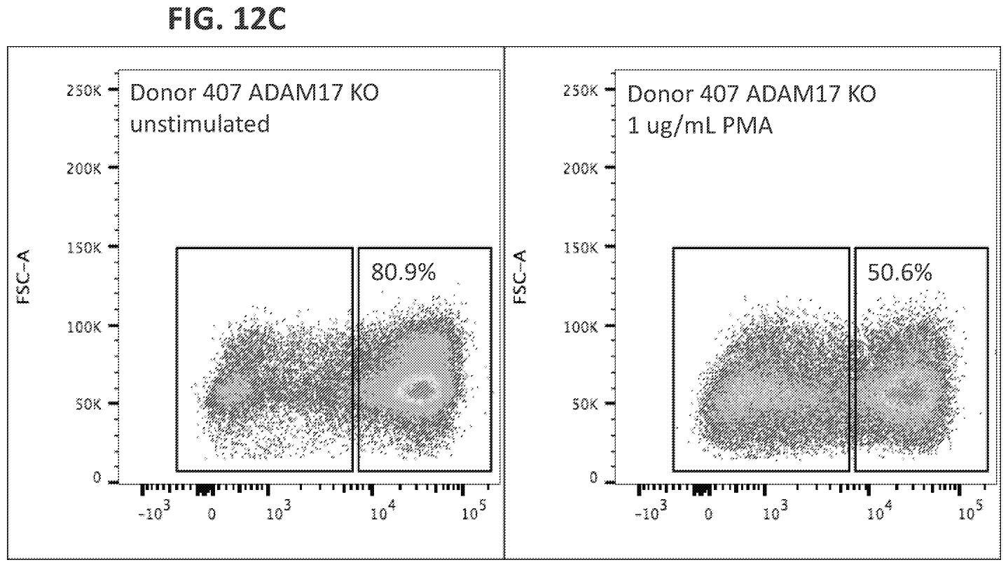

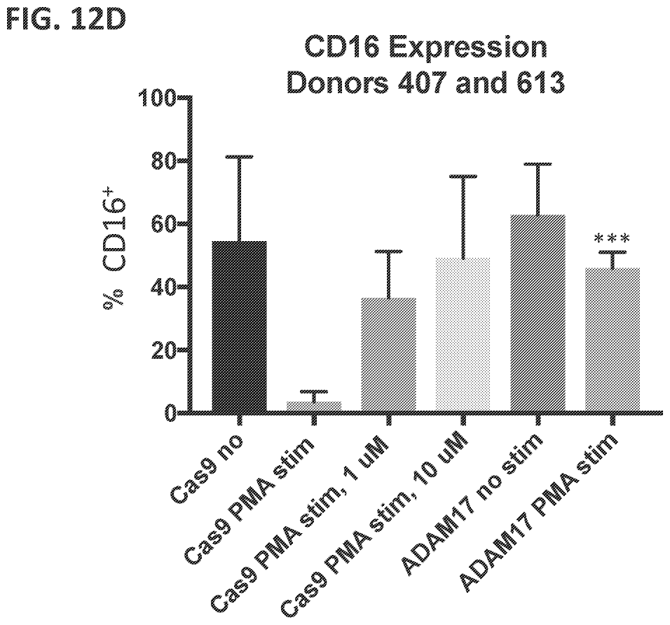

3×10⁶ NK cells from two donors (donors 407 and 613) stimulated with aAPCs were electroporated using the optimized NEON electroporation conditions with 10 μg Cas9 mRNA and 10 μg gRNA. Five days after electroporation, cells were treated for 1 hour with ADAM17 inhibitor or DMSO. Cells were then stimulated for 1 hour with 1 μg/mL PMA or left unstimulated. Cells were harvested and stained with antibodies for CD56, CD3, CD16, and SYTOX Blue viability dye. Cells were gated on single cells (FSC-H vs FSC-A), CD56⁺, CD3⁻, SYTOX Blue⁻, and CD16⁺. CD16 expression in PMA stimulated ADAM17 knockout cells was compared to PMA stimulated matched donor samples electroporated with Cas9 mRNA alone. Results are shown in FIG. 12.

### Example 3

Unless otherwise indicated, the materials and methods of Example 1 were used; however, the optimized NEON electroporation conditions described in Example 2 was used for Example 3.

### Example 3A

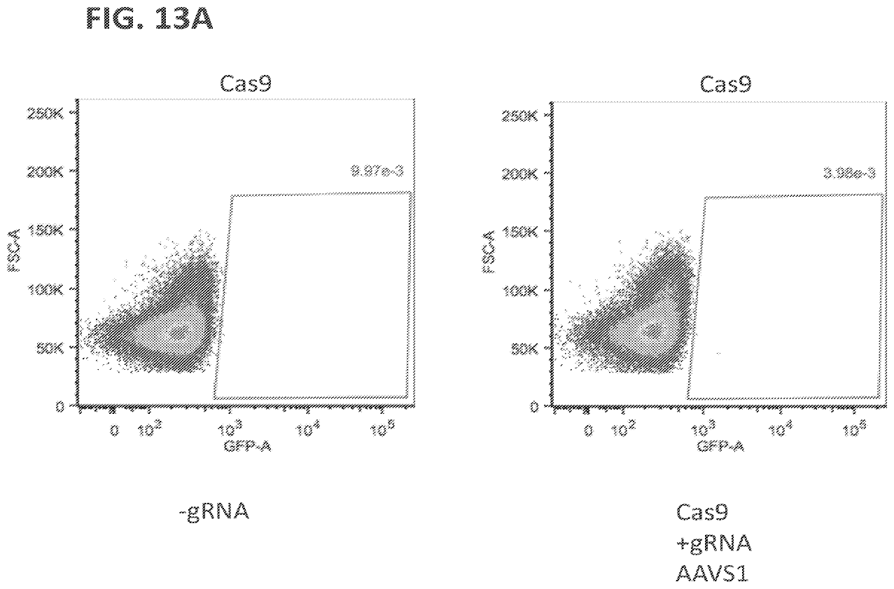

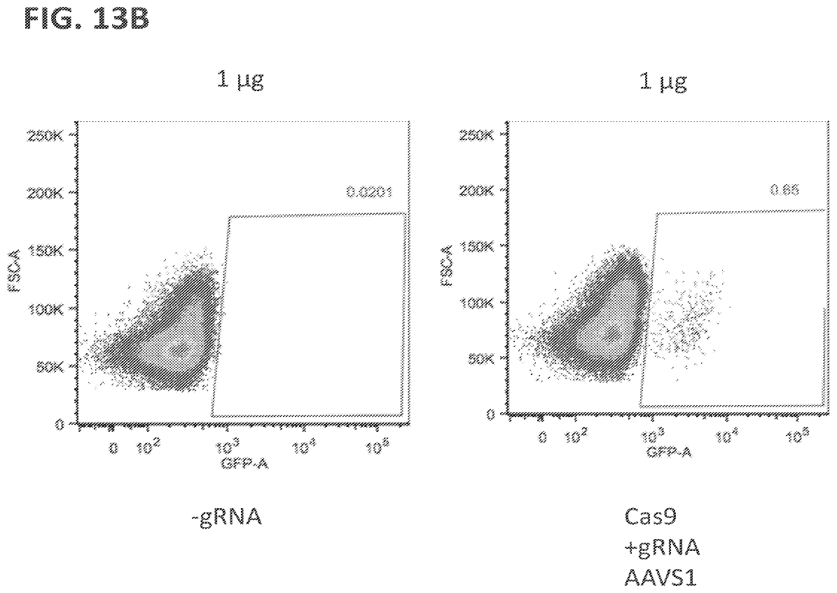

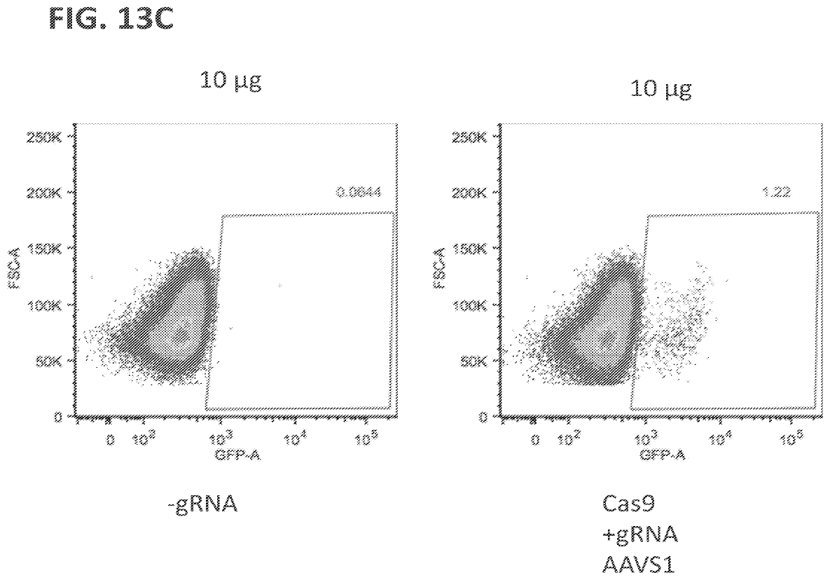

NK cells purified from peripheral blood mononuclear cells were co-cultured with artificial antigen presenting cells (aAPC) expressing membrane bound IL-21 (2:1 aAPC:NK) ratio. On day 7 post-culture, NK cells were electroporated with the optimized NEON electroporation conditions (as described in Example 2), to measure homologous recombination using a donor vector that expresses GFP after integration at AAVS1 (Doggybone Splice-Acceptor GFP). 3 million NK cells were electroporated with the indicated amount of vector DNA alone or with 15 μg of Cas9 and 15 μg AAVS1 gRNA. 15 μg of Cas9 alone or with AAVS1 gRNA served as the donor negative controls. Five days later, GFP expression in viable cells was measured by flow cytometry. Results are shown in FIG. 13.

### Example 3B

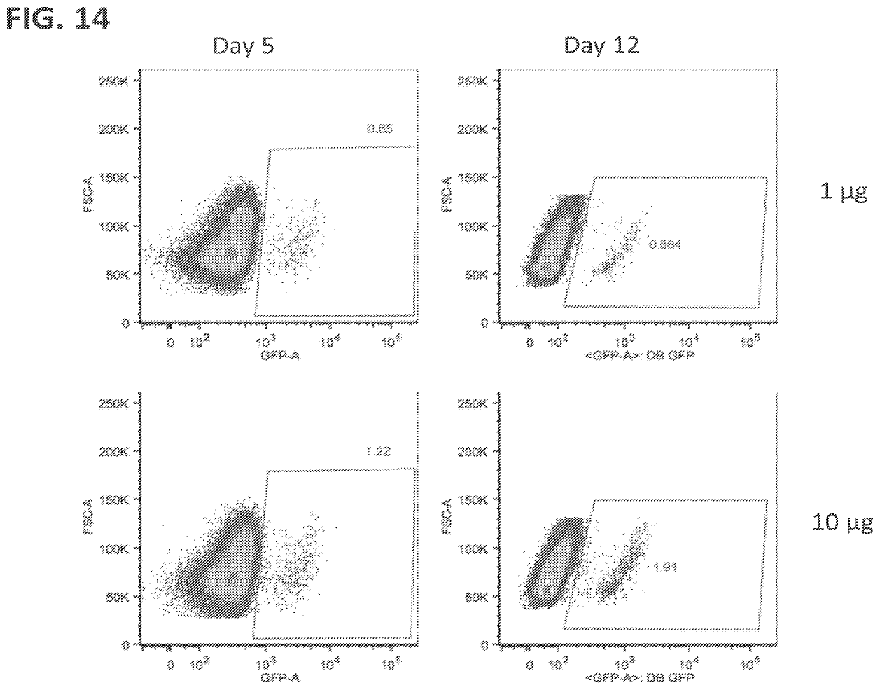

NK cells were treated as described in Example 3A. On day 5 post-electroporation, cells were collected for flow cytometric analysis or co-cultured with aAPC (1:1 ratio) for 7 days. After the second culture with aAPC, GFP expression was measured in a subset of cells to confirm that the donor GFP was stably integrated into the NK cells (Day 12). Results are shown in FIG. 14.

### Example 3C

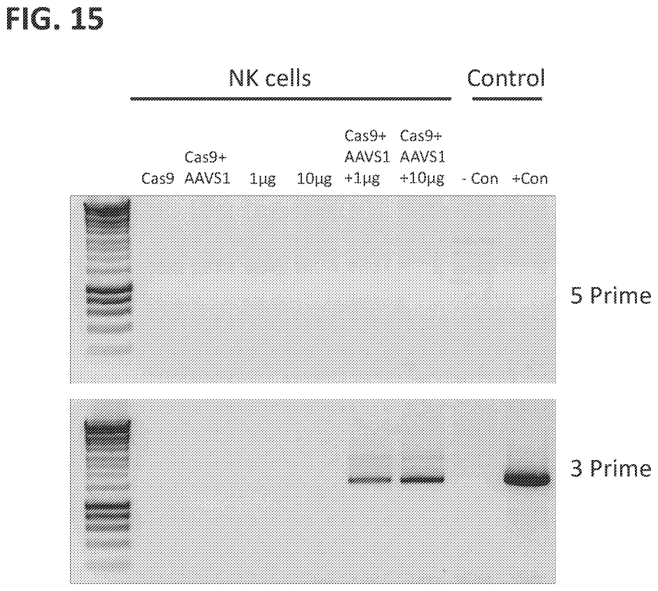

NK cells were treated as described in Example 3A. On day 5 post-electroporation, cells were collected for flow cytometric analysis or co-cultured with aAPC (1:1 ratio) for 7 days. To confirm integration, gDNA was collected from electroporated NK cells. The gDNA was PCR amplified using primers that target from the AAVS1 site into donor sequences (5 prime) or from the donor seqeunces into AAVS1 (3 prime). Results are shown in FIG. 15.

### Example 3D

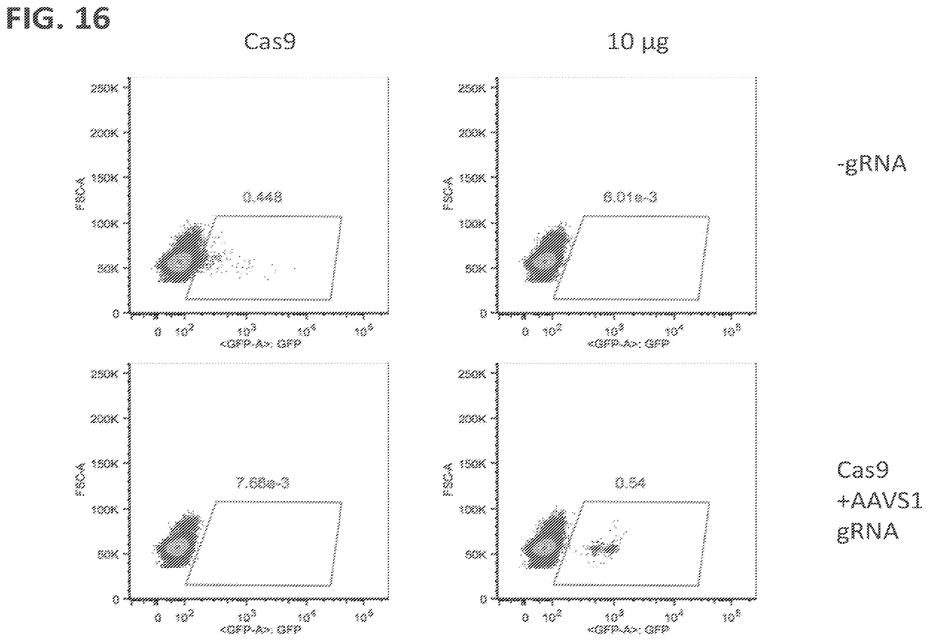

NK cells were treated as described in Example 3A except a different donor vector (10 μg) that also expresses GFP when integrated into the AAVS1 target site was used (Minicircle Splice Acceptor GFP). On day 5 post-electroporation, cells were co-cultured with aAPC (1:1 ratio) for 7 days. After the second culture with aAPC, GFP expression was measured in a subset of cells to confirm that the donor GFP was stably integrated into the NK cells. Results are shown in FIG. 16.

### Example 4

NK cell function following gene editing will be assessed in vitro and in vivo using standard techniques. In vitro proliferation and viability will be measured by CellTrace Violet Cell Proliferation Dye (Invitrogen) and by viability dye as above. NK cytokine production, cytoxicity and ADCC will be measured by co-culturing NK cells with cytokines or different target pediatric cancer cell lines including RAH, HOS, MG-63, and adult K562 cells.INFγ and TNFα cytokine production following stimulation with IL-12/IL-18 or co-culture with cancer cell lines will be measured by intracellular cytokine staining (BD Bioscience) via flow cytometry and in the culture supernatant by ELISA (R&D System). Cytotoxicity will be assessed by CD107a (BD Bioscience) expression on NK cells and killing of fluorescently-la.beled target cells by flow cytometry and/or by chromium release assay. ADCC will be determined by co-culturing NK cells, RAH cells and rituximab (specific for CD20) and assessing the killing of the RAH target cells. NK cell survival, proliferation and cytotoxicity will be measured in vivo using the NSG mouse model for the most promising gene edited combinations identified in vitro (Vallera et al., Clinical Cancer Research, Feb. 4, 2016, clincanres.2710.2015; Vallera et al., Clinical Cancer Research, 2016, 22(14):3440-3450). Groups will include, RAH-luc alone, RAJI-luc plus unedited NK, RAJI-luc plus unedited NK and IL15, RAJI-luc plus edited NK, RAJI-luc plus edited NK and IL15. NK cell survival and proliferation will be measured by flow cytometry. The ability of edited NK cells to eliminate cancer cells in vivo will be determined by measuring tumor burden following NK cell treatment of the NSG mice injected with a luciferase expressing tumor cell line (for example, RAJI-luc). Tumor burden will be measured by assessing luciferase expression in treated mice.

The ability of edited NK cells to eliminate cancer cells in vivo is expected to increase when edited NK cells exhibit increased activity in vitro,

### Example 5

In some cases, NK cells will be selected following gene editing. Edited NK cells may be selected using one or more of the following methods: flow sorting using GFP expression; magnetic bead separation targeting a cell-surface marker; transient drug resistance gene expression (for example, Puromycin). Following selection, the edited NK cells may be expanded using the aAPC clone 9 with membrane-bound IL-21.

For example, NK cells modified at CD16 to prevent ADAM17 mediated cleavage may be selected in the following manner. Three to five days after targeting CD16 with Cas9-gRNA-oligo to make it cleavage resistant, NK cells will be treated with ADAM17. ADAM17 treatment cleaves CD16 on non-modified NK cells while the modified NK cells retain CD16. The modified NK cells may then be separated using antibodies against CD16. This selection process may be used in CD16-modified cells alone or in conjunction with another editing event such as knocking out an inhibitory receptor.

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method comprising editing a genome of a stimulated primary natural killer (NK) cell, wherein the primary NK cell comprises a CD3⁻CD56⁺ cell.

2. The method of claim 1, the method comprising electroporation of the primary NK cell.

3. The method of claim 2, wherein electroporation of the primary NK cell comprises exposing the primary NK cell to at least 1700 volts and up to 2000 volts and at least 2 energy pulses, wherein at least one energy pulse has a duration of at least 2 milliseconds.

4. The method of claim 1, the method comprising introducing a nuclease or a nucleic acid encoding a nuclease into the primary NK cell.

5. The method of claim 4, wherein the nuclease comprises Cas9.

6. The method of claim 1, the method comprising introducing a chemically modified guide RNA (gRNA) into the primary NK cell.

7. The method of claim 6, wherein the chemically modified gRNA comprises 2′-O-methyl (M), 2′-O-methyl-3′-phosphorothioate (MS), or 2′-O-methyl-3′-thiophosphonoacetate (MSP).

8. The method of claim 1, the method further comprising exposing a primary NK cell to a cytokine to produce a stimulated primary NK cell.

9. The method of claim 8, wherein the cytokine comprises a cytokine bound to an artificial antigen presenting cell (aAPC).

10. The method of claim 1, wherein editing the genome comprises editing a gene for an activating receptor, an inhibitory receptor, an adaptor molecule, a downstream signaling molecule, a component of a cytotoxic granule, a cytokine, a chemokine, a cytokine receptor, or a chemokine receptor, or a combination thereof.

11. A genome-edited primary NK cell, wherein the genome-edited primary NK cell comprises a CD56⁺CD3⁻ cell.

12. The genome-edited primary NK cell of claim 11, wherein a gene is deleted.

13. The genome-edited primary NK cell of claim 11, wherein a gene comprises a point mutation.

14. The genome-edited primary NK cell of claim 11, the genome-edited primary NK cell comprising an exogenous gene.

15. The genome-edited primary NK cell of claim 11, wherein the genome-edited primary NK cell comprises a modification that alters expression or activity of an activating receptor, an inhibitory receptor, an adaptor molecule, a downstream signaling molecule, a component of a cytotoxic granule, a cytokine, a chemokine, a cytokine receptor, or a chemokine receptor, or a combination thereof.

16. The genome-edited primary NK cell of claim 11, wherein the genome-edited primary NK cell exhibits at least one of increased stimulation-induced cytokine production, increased capacity to kill cancer cells, increased survival, and increased capacity to expand relative to a non-genome-edited primary NK cell.

17. The genome-edited primary NK cell of claim 11, wherein the genome-edited primary NK cell exhibits increased expression of an activating receptor relative to a non-genome-edited primary NK cell.

18. The genome-edited primary NK cell of claim 11, wherein the genome-edited primary NK cell exhibits decreased expression of an inhibitory receptor relative to a non-genome-edited primary NK cell.

19. A method for treating or preventing a disease in a subject, the method comprising:
administering to the subject a composition comprising the genome-edited primary NK cell of claim 11.

20. The method of claim 19, wherein the disease comprises cancer, a precancerous condition, an infection with a pathogen, or a viral infection.


SEQUENCE LISTING (34 sequences)

SEQ ID NO: 1 - DNA, 20 bp, artificial
  Guide RNA target sequence
ggcagcatagtgagcccaga

SEQ ID NO: 2 - DNA, 20 bp, artificial
  Guide RNA target sequence
cctgctcgtggtgaccgaag

SEQ ID NO: 3 - DNA, 20 bp, artificial
  Guide RNA target sequence
gaaccacgctggtcaggaat

SEQ ID NO: 4 - DNA, 20 bp, artificial
  Guide RNA target sequence
gacctgggtcacttgtgccg

SEQ ID NO: 5 - DNA, 20 bp, artificial
  Guide RNA target sequence
gggttccattacggccagcg

SEQ ID NO: 6 - DNA, 20 bp, artificial
  Guide RNA target sequence
aacaactatcgttaccacag

SEQ ID NO: 7 - DNA, 20 bp, artificial
  Guide RNA target sequence
tctcatcattctttccacct

SEQ ID NO: 8 - DNA, 20 bp, artificial
  Guide RNA target sequence
aggtggaaagaatgatgaga

SEQ ID NO: 9 - DNA, 20 bp, artificial
  Guide RNA target sequence
gtcaccaatcctgtccctag

SEQ ID NO: 10 - RNA, 20 nt, artificial
  Guide RNA sequence
ggcagcauagugagcccaga

SEQ ID NO: 11 - RNA, 20 nt, artificial
  Guide RNA sequence
ccugcucguggugaccgaag

SEQ ID NO: 12 - RNA, 20 nt, artificial
  Guide RNA sequence
gaaccacgcuggucaggaau

SEQ ID NO: 13 - RNA, 20 nt, artificial
  Guide RNA sequence
gaccugggucacuugugccg

SEQ ID NO: 14 - RNA, 20 nt, artificial
  Guide RNA sequence
ggguuccauuacggccagcg

SEQ ID NO: 15 - RNA, 20 nt, artificial
  Guide RNA sequence
aacaacuaucguuaccacag

SEQ ID NO: 16 - RNA, 20 nt, artificial
  Guide RNA sequence
ucucaucauucuuuccaccu

SEQ ID NO: 17 - RNA, 20 nt, artificial
  Guide RNA sequence
agguggaaagaaugaugaga

SEQ ID NO: 18 - RNA, 20 nt, artificial
  Guide RNA sequence
gucaccaauccugucccuag

SEQ ID NO: 19 - DNA, 21 bp, artificial
  Primer sequence
gcacagggtggaacaagatgg

SEQ ID NO: 20 - DNA, 21 bp, artificial
  Primer sequence
caccaccccaaaggtgaccgt

SEQ ID NO: 21 - DNA, 20 bp, artificial
  Primer sequence
gggtgaaggctcttagtagg

SEQ ID NO: 22 - DNA, 19 bp, artificial
  Primer sequence
caggctctctttgatctgc

SEQ ID NO: 23 - DNA, 20 bp, artificial
  Primer sequence
cccgatgtgagcagttttcc

SEQ ID NO: 24 - DNA, 20 bp, artificial
  Primer sequence
gagacaggcccatctccttt

SEQ ID NO: 25 - DNA, 20 bp, artificial
  Primer sequence
gaggagcaacaggatggact

SEQ ID NO: 26 - DNA, 20 bp, artificial
  Primer sequence
tgcagtgtttcaggattgca

SEQ ID NO: 27 - DNA, 20 bp, artificial
  Primer sequence
cttctgcgtacaaagggctg

SEQ ID NO: 28 - DNA, 20 bp, artificial
  Primer sequence
gagagtctgatgggagaggc

SEQ ID NO: 29 - DNA, 20 bp, artificial
  Primer sequence
caatgggagatgagggtttg

SEQ ID NO: 30 - DNA, 24 bp, artificial
  Primer sequence
caatgagaactctattccctgaaa

SEQ ID NO: 31 - DNA, 20 bp, artificial
  Primer sequence
ccccaccattcctaccactt

SEQ ID NO: 32 - DNA, 20 bp, artificial
  Primer sequence
ccccaccattcctaccactt

SEQ ID NO: 33 - DNA, 20 bp, artificial
  Primer sequence
actcctttcatttgggcagc

SEQ ID NO: 34 - DNA, 20 bp, artificial
  Primer sequence
ggttctggcaaggagagaga